(19) United States
(12) Patent Application Publication
(10) Pub. No.: US 2005/0142589 A1
(43) Pub. Date: Jun. 30, 2005

(54) Oligonucleotide probe sets and uses thereof

(76) Inventors: Ron S. Broide, San Marcos, CA (US); Alain Trembleau, Yerres (FR); Warren G. Young, San Diego, CA (US); John H. Morrison, Rye, NY (US); Floyd Bloom, San Diego, CA (US)

Correspondence Address: DAVID R PRESTON & ASSOCIATES, 12625 HIGH BLUFF DRIVE, SUITE 205, SAN DIEGO, CA, 92130, US

(21) Appl. No.: 10/970,760

(22) Filed: Oct. 21, 2004

Related U.S. Application Data
(60) Provisional application No. 60/513,856, filed on Oct. 22, 2003.

(51) Int. Cl. C12Q 1/68

(52) U.S. Cl. 435/6

(57) ABSTRACT

The present invention relates to methods and probes for detecting relative levels of multiple nucleic acid sequences of interest. The invention includes a method to detect the relative amounts of at least two target nucleic acid sequences in at least one sample by use of a corresponding set of detectably labelled oligonucleotide probes for each of the at least two target nucleic acid sequences, and detecting the hybridization of each of the corresponding sets of oligonucleotide probes to its respective target nucleic acid sequence. The invention also includes a method to detect the relative amounts of multiple target messenger RNAs in at least one microscopy sample by in situ hybridization using sets of oligonucleotide probes. Also disclosed are methods to provide corresponding sets of oligonucleotide probes for target nucleic acid sequences.

The present application claims priority from U.S. Provisional Patent Application No. 60/513,856 to Broide et al., entitled “Standardized quantitative in situ hybridization using radioactive oligonucleotide probes for detecting relative levels of mRNA transcripts verified by Real-Time PCR”, filed on 22 Oct. 2003, which is incorporated by reference herein in its entirety.

### BACKGROUND OF THE INVENTION

1. Field of the Invention

The present invention relates generally to molecular biology, and more specifically to methods and probes for detecting relative levels of multiple nucleic acid sequences of interest.

2. Background

Quantitative comparison of more than a single target nucleic acid sequence (e.g., messenger RNAs from two or more different gene products) is sometimes desirable. Molecular techniques such as Northern blot analysis, RNase protection analysis, and quantitative PCR offer some possible approaches for comparison of relative levels of target nucleic acid sequences. However, these approaches give limited information regarding the cellular or tissue distribution of such targets.

In situ hybridization (ISH) is a technique useful for obtaining information on anatomical distribution of target messenger RNAs. For example, ISH is a widely used technique in neuroscience for mapping gene expression in the brain. A number of ISH protocols utilize either cRNA riboprobes or DNA oligonucleotide probes for detecting low to high abundance mRNAs. See, for example, Cloez-Tayarani and Fillion (1997) Brain Res. Brain Res. Protoc., 1: 195-202, Erdtmann-Vourliotis et al. (1999) Brain Res. Brain Res. Protoc,. 4: 82-91, Key et al. (2001) Brain Res. Brain Res. Protoc., 8: 8-15, C. Le Moine, “Quantitative in situ hybridization using radioactive probes to study gene expression in heterocellular systems”, in I. A. Darby (editor), “In situ hybridization protocols”, Humana Press, Totowa, N.J., 2000, pp. 143-156, Newton et al. (2002) Brain Res. Brain Res. Protoc., 9: 214-219, Trembleau et al. (1993) J. Histochem. Cytochem., 41: 489-498, Vizi et al. (2001) Brain Res. Brain Res. Protoc., 8: 32-44, Winzer-Serhan et al. (1999) Brain Res. Brain Res. Protoc., 3: 229-241, which are incorporated by reference in their entirety herein. However, these ISH techniques lack the necessary standardization for quantitative comparisons between different mRNA transcripts in different anatomical areas or across experimental conditions. Further improvements in the effectiveness and sensitivity of detecting and comparing relative levels of target nucleic acid sequences are desirable. The present invention addresses the existing problems and provides related benefits.

### BRIEF SUMMARY OF THE INVENTION

The present invention recognizes that it can be desirable to detect and compare relative levels of multiple target nucleic acid sequences, such as multiple mRNAs, in a sample. The present invention provides a novel method to detect and compare such target nucleic acid sequences, and, unlike existing technologies, the present invention further provides the capability to obtain information about the cellular or tissue distribution of such targets.

Thus, one aspect of the present invention includes a method to detect the relative amounts of at least two target nucleic acid sequences in at least one sample by use of a corresponding set of detectably labelled oligonucleotide probes for each of the at least two target nucleic acid sequences, and detecting the hybridization of each of the corresponding sets of oligonucleotide probes to its respective target nucleic acid sequence.

Another aspect of the present invention is a method to detect the relative amounts of at least two target messenger RNAs in at least one microscopy sample, by use of a corresponding set of oligonucleotide probes for each of the at least two target messenger RNAs, and detecting the in situ hybridization of each of corresponding set of oligonucleotide probes to its respective target messenger RNA. One embodiment of this aspect of the present invention is a standardized quantitative in situ hybridization (SQuISH™) method that utilizes a known number of multiple oligonucleotide probes for accurate comparison of relative mRNA levels from two or more mRNA transcripts in at least one tissue or cell sample. The SQuISH™ method can be used to quantitatively compare relative levels of two or more mRNAs in discrete anatomical or cellular locations, and provides the additional benefits of increasing sensitivity while decreasing non-specific hybridization.

Yet another aspect of the present invention is a method to provide corresponding sets of oligonucleotide probes for target nucleic acid sequences.

These aspects of the invention, as well as others described herein, can be achieved by using the methods, articles of manufacture and compositions of matter described herein. To gain a full appreciation of the scope of the present invention, it will be further recognized that various aspects of the present invention can be combined to make additional, desirable embodiments of the invention.

### BRIEF DESCRIPTION OF THE DRAWINGS

FIG. 1 depicts a schematic diagram of the IRSp53 (FIG. 1A, GenBank accession number AF390179) and Calsenilin (FIG. 1B, GenBank accession number AF184624) messenger RNA transcripts (light grey lines) along with an alignment of their respective probes (heavy black lines), as described in detail in Example 1. The two splice variants for IRSp53 are indicated as svA and svB, with svA containing an additional 120 base-pair fragment (dark grey line). The splice variants for Calsenilin mRNA are not shown. Alignment of the riboprobes (RP), oligonucleotide probes (01-6) and PCR forward (RTf) and reverse (RTr) primers are indicated for each target messenger RNA; sequences for the probes and primers are given in Table 1 (see Example 1).

FIG. 2 depicts electrophoretic analysis of oligonucleotide probes for IRSp53 and Calsenilin messenger RNAs. Bands for two random probes (IRSp53 oligonucleotide probe 5, in the lane marked “IRSp53”, SEQ ID No. 6, and Calsenilin oligonucleotide probe 1, in the lane marked “Calsenilin”, SEQ ID No. 11) are shown, along with DNA size markers. The asterisk indicates the likely position of a 40-mer unlabelled oligonucleotide.

FIG. 3 depicts the relative amounts of IRSp53 and Calsenilin messenger RNAs as seen in expression patterns obtained by autoradiography of mouse brain microscopy samples, as described in Example 1. Hybridization signals from oligonucleotide probes (FIG. 3A and FIG. 3B) and riboprobes (FIG. 3C and FIG. 3D) are shown in these autoradiographic images of adjacent brain sections at the level of the basal ganglia. Abbreviations: ACB, nucleus accumbens; CP, caudate putamen; Ctx, cortex. Size bar=1 millimeter.

FIG. 4 depicts the relative amounts of IRSp53 and Calsenilin messenger RNAs as seen in expression patterns obtained by autoradiography of mouse brain microscopy samples, as described in Example 1. Hybridization signals from oligonucleotide probes (FIG. 4A and FIG. 4B) and riboprobes (FIG. 4C and FIG. 4D) are shown in these autoradiographic images of adjacent brain sections at the level of the hippocampus. Abbreviations: Ctx, cortex; Hi, hippocampus; Hy, hypothalamus; Th, thalamus. Size bar=1 millimeter.

### DETAILED DESCRIPTION OF THE INVENTION

As a non-limiting introduction to the breadth of the present invention, the present invention includes several general and useful aspects, including:

1. A method to detect the relative amounts of at least two target nucleic acid sequences in at least one sample, including providing at least one sample suspected of containing the at least two target nucleic acid sequences; providing a corresponding set of oligonucleotide probes for each of the at least two target nucleic acid sequences, wherein each of the corresponding sets of oligonucleotide probes includes N oligonucleotide probes, and wherein each of the oligonucleotide probes includes a sequence of X bases and at least one detectable label; contacting the at least one sample with the corresponding sets of oligonucleotide probes so that each of the at least two target nucleic acid sequences is contacted with the corresponding set of oligonucleotide probes; incubating the at least one sample and the corresponding sets of oligonucleotide probes under conditions that allow hybridization of each of the target nucleic acid sequences, if present in the at least one sample, to the corresponding set of oligonucleotide probes; and detecting the hybridization, wherein the detecting of the hybridization indicates the relative amounts of each of the at least two target nucleic acid sequences in the at least one sample.

2. A method to detect the relative amounts of at least two target messenger RNAs in at least one microscopy sample, including providing at least one microscopy sample suspected of containing the at least two target messenger RNAs; providing a corresponding set of oligonucleotide probes for each of the at least two target messenger RNAs, wherein each of the corresponding sets of oligonucleotide probes comprises N oligonucleotide probes, where N comprises between about 2 to about 20 oligonucleotide probes, and wherein each of the oligonucleotide probes comprises a sequence comprising X bases, where X comprises between about 15 to about 60 bases, and comprising a GC content of between about 40% to about 65%, and permitting a spacing of at least about 15 bases between adjacent oligonucleotide probes when the adjacent oligonucleotide probes are hybridized to the corresponding the target messenger RNA; and at least one detectable label; contacting the at least one microscopy sample with the corresponding sets of oligonucleotide probes so that each of the at least two target messenger RNAs is contacted with the corresponding set of oligonucleotide probes; incubating the at least one microscopy sample and the corresponding sets of oligonucleotide probes under conditions that allow in situ hybridization of each of the target messenger RNAs, if present in the at least one microscopy sample, to the corresponding set of oligonucleotide probes; and detecting said in situ hybridization, wherein the detecting of said in situ hybridization indicates the relative amounts of each of the at least two target messenger RNAs in the at least one microscopy sample.

3. A method to provide sets of oligonucleotide probes useful in detecting at least two target nucleic acid sequences, including the steps of: (a) selecting said at least two target nucleic acid sequences; (b) designing a corresponding set of oligonucleotide probes for each of the at least two target nucleic acid sequences, wherein each of the corresponding sets of oligonucleotide probes includes N oligonucleotide probes, where N includes between about 2 to about 24 oligonucleotide probes, and wherein each of the oligonucleotide probes includes (i) a sequence complementary to at least part of the corresponding target nucleic acid sequence, and including X bases, where X includes between about 15 to about 60 bases, and including a GC content of between about 40% to about 65%, and permitting a spacing of at least about 15 bases between adjacent oligonucleotide probes when the adjacent oligonucleotide probes are hybridized to the corresponding target nucleic acid sequence; and (ii) at least one detectable label; and (c) synthesizing the designed corresponding sets of oligonucleotide probes for each of the at least two target nucleic acid sequences.

Further objectives and advantages of the present invention will become apparent as the description proceeds and when taken in conjunction with the accompanying drawings. To gain a full appreciation of the scope of the present invention, it will be further recognized that various aspects of the present invention can be combined to make desirable embodiments of the invention.

Throughout this application various publications are referenced. The disclosures of these publications are hereby incorporated by reference, in their entirety, in this application. Citations of these documents are not intended as an admission that any of them are pertinent prior art. All statements as to the date or representation as to the contents of these documents is based on the information available to the applicant and does not constitute any admission as to the correctness of the dates or contents of these documents.

Unless defined otherwise, all technical and scientific terms used herein have the same meaning as commonly understood by one of ordinary skill in the art to which this invention belongs. Generally, the nomenclature used herein and the manufacture or laboratory procedures described below are well known and commonly employed in the art. The technical terms used herein have their ordinary meaning in the art that they are used, as exemplified by a variety of technical dictionaries. Where a term is provided in the singular, the inventors also contemplate the plural of that term. The nomenclature used herein and the procedures described below are those well known and commonly employed in the art. Where there are discrepancies in terms and definitions used in references that are incorporated by reference, the terms used in this application shall have the definitions given herein. Other technical terms used herein have their ordinary meaning in the art that they are used, as exemplified by a variety of technical dictionaries (for example, Chambers Dictionary of Science and Technology, Peter M. B. Walker (editor), Chambers Harrap Publishers, Ltd., Edinburgh, UK, 1999, 1325 pp.). The inventors do not intend to be limited to a mechanism or mode of action. Reference thereto is provided for illustrative purposes only.

I. Method to Detect at Least Two Target Nucleic Acid Sequences

The present invention includes a method to detect the relative amounts of at least two target nucleic acid sequences in at least one sample, including providing at least one sample suspected of containing the at least two target nucleic acid sequences; providing a corresponding set of oligonucleotide probes for each of the at least two target nucleic acid sequences, wherein each of the corresponding sets of oligonucleotide probes includes N oligonucleotide probes, and wherein each of the oligonucleotide probes includes a sequence of X bases and at least one detectable label; contacting the at least one sample with the corresponding sets of oligonucleotide probes so that each of the at least two target nucleic acid sequences is contacted with the corresponding set of oligonucleotide probes; incubating the at least one sample and the corresponding sets of oligonucleotide probes under conditions that allow hybridization of each of the target nucleic acid sequences, if present in the at least one sample, to the corresponding set of oligonucleotide probes; and detecting the hybridization, wherein the detecting of the hybridization indicates the relative amounts of each of the at least two target nucleic acid sequences in the at least one sample.

The method of the present invention may be applied to any at least one suitable sample that is suspected of containing the at least two target nucleic acid sequences of interest. The sample may be of entirely natural origin, of entirely non-natural origin (such as of synthetic origin), or a combination of natural and non-natural origins. A sample may include cell fragments, whole cells (such as prokaryotic cells, bacterial cells, eukaryotic cells, plant cells, fungal cells, or cells from multi-cellular organisms including invertebrates, vertebrates, mammals, and humans), tissues, organs, or biological fluids (such as, but not limited to, blood, serum, plasma, urine, semen, and cerebrospinal fluid). A sample may be an extract made from biological materials, such as from prokaryotes, bacteria, eukaryotes, plants, fungi, multi-cellular organisms or animals, invertebrates, vertebrates, mammals, non-human mammals, and humans. A sample may be an extract made from whole organisms or portions of organisms, cells, organs, tissues, fluids, whole cultures or portions of cultures, or environmental samples or portions thereof. A sample may be a product of an amplification reaction, such as, but not limited to, a polymerase chain reaction product, a reverse transcriptase amplification product, or other nucleic acid amplification methods known in the art (Andras et al. (2001) Mol. Biotechnol., 19: 29-44, which is incorporated by reference in its entirety herein). A sample may include a nucleic acid located in situ within a cell or a tissue, such as, but not limited to, an in situ amplified nucleic acid (Long (1998) Eur. J. Histochem., 42: 101-109, which is incorporated by reference in its entirety herein), or a chromosome, plasmid, or other cellular structure that contains a nucleic acid (Lichter et al. (1990), Science, 247: 64-69, which is incorporated by reference in its entirety herein). A sample may need minimal preparation (for example, collection into a suitable container) for use in a method of the present invention, or more extensive preparation (including any combination of steps such as, but not limited to, removal, inactivation, or blocking of undesirable material, such as contaminants, undesired nucleic acids, or endogenous enzymes; filtration, size selection, affinity purification, concentration, or dilution; nucleic acid isolation, purification, amplification, denaturation, electrophoresis, or attachment to a solid substrate such as a membrane, chip, or particle; cell lysis, tissue digestion or permeabilization; chromosome preparation and spreading; and cell or tissue fixation, embedding, sectioning, staining, or other preparation for microscopy). The method of the present invention can be applied to samples that are included as part of a membrane blotting procedure (for example, Northern blots or Southern blots) or other nucleic acid hybridization procedures (for example, nuclease protection assays, hybridization assays in solution, hybridization assays on solid substrates such as chips or particles, or hybridization assays with nucleic acid arrays). The method of the present invention can be applied to samples that are included as part of in situ hybridization, molecular diagnostics, genetic screens, and other practical applications.

The method of the present invention may be applied to at least two target nucleic acid sequences of interest. Suitable target nucleic acid sequences can include any type of nucleic acid, for example, DNA or RNA, or a nucleic acid mimic (such as, but not limited to, a peptide nucleic acid), or a combination thereof. In one embodiment of the method, the target nucleic acid sequences can include messenger RNAs (mRNAs) or RNA transcripts. In another embodiment of the method, the target nucleic acid sequences can include genomic DNA. In yet another embodiment of the method, the target nucleic acid sequences can include nucleic acid constructs such as plasmids optionally including inserted sequences.

The method of the present invention includes the step of providing a corresponding set of oligonucleotide probes for each of the at least two target nucleic acid sequences. Thus, a corresponding set of oligonucleotide probes is provided for each individual target nucleic acid sequence. Each corresponding set of oligonucleotide probes includes N oligonucleotide probes, wherein N is an integer greater than 1. Preferably N ranges from about 2 to about 24, or from about 2 to about 20, or from about 2 to about 14, or from about 2 to about 12 or from about 3 to about 10, or from about 4 to about 10. Preferably, the value of N is identical between corresponding sets of oligonucleotide probes. For example, in an experiment designed to study two target mRNAs, two corresponding sets of oligonucleotide probes are provided, wherein one set of oligonucleotide probes corresponds specifically to one of the target mRNAs, and wherein the two corresponding sets of oligonucleotide probes each includes an identical number N of oligonucleotide probes (for example, the two corresponding sets of oligonucleotide probes can each consist of six probes). In some embodiments of the method, N may not be identical between corresponding sets of oligonucleotide probes, and the differing numbers of oligonucleotide probes in each corresponding set must be taken into account if accurate quantification of the amounts of each of the at least two target nucleic acid sequences is desired.

N, the number of oligonucleotide probes per set, is preferably selected according to the expected abundance or scarcity of the target nucleic acid sequence in the at least one sample. As a general practice, the less abundant the target nucleic acid sequence is, the larger N preferably is. In one embodiment where the target nucleic acid sequences are messenger RNAs, the messenger RNAs can be predicted or expected to be characterized by “low copy number” (about 1 to about 30 copies per cell), “medium copy number” (about 30 to about 1000 copies per cell), or “high copy number” (greater than about 1000 copies to about 100,000 copies per cell). In this non-limiting embodiment, typical values of N for these ranges can be from about 3 to about 4 oligonucleotide probes for “high copy number” messenger RNAs, from about 5 to about 8 probes for “medium copy number” messenger RNAs, and from about 9 to about 12 probes for “low copy number” messenger RNAs. However, these values of N merely serve as guidelines for this embodiment, and the actual N may be varied by the user according to his or her requirements.

Each of the oligonucleotide probes is designed to include a sequence that is complementary to at least part of the corresponding target nucleic acid sequence. By “complementary” is meant that stable hydrogen bonding occurs between a purine base and a pyrimidine base according to Watson-Crick base-pairing rules, such as is seen in double-stranded naturally occurring nucleic acids where the pair of bases consists of a purine base (adenine or guanine) on one strand of nucleic acid and a pyrimidine base (thymine, cytosine, or uracil) on a second and opposite-running strand of nucleic acid. According to Watson-Crick base-pairing rules, adenine base-pairs with thymine (in deoxyribonucleic acids) or with uracil (in ribonucleic acids), and guanine base-pairs with cytosine. Analogous complementary base-pairing may also occur between bases of a nucleic acid mimic (such as, but not limited to, a peptide nucleic acid) and nucleotides of a naturally occurring nucleic acid. Each of the oligonucleotide probes most preferably includes a sequence that is specifically complementary to at least part of the corresponding target nucleic acid sequence. By “specifically complementary” is meant that the oligonucleotide probe's sequence is complementary to its intended target nucleic acid sequence, and preferably does not show substantial similarity or homology to nucleic acid sequences that are not of interest. Thus, each oligonucleotide sequence is preferably compared against known sequences, such as, but not limited to, sequences publicly available in the databases maintained by the National Center for Biotechnology Information (accessible on-line at www.ncbi.nlm.nih.gov), to confirm that the oligonucleotide sequence is specifically complementary to the target nucleic acid sequence, and preferably does not show substantial similarity or homology to other known nucleic acid sequences.

Each of the oligonucleotide probes includes a sequence of X bases (or nucleotides) and at least one detectable label, wherein X is an integer. Preferably X ranges from about 15 to about 60 bases, or from about 20 to about 50 bases, or about 20 to about 40 bases. In a given corresponding set of oligonucleotide probes, X is preferably identical for all the probes in order to minimize variability in probe length, probe GC content, probe annealing and melting temperatures, and other factors that influence hybridization behaviour. In some embodiments, particularly where the probes are longer probes, X may vary slightly between the probes of a given corresponding set of oligonucleotide probes, if such variation does not result in substantial unwanted variability in hybridization behaviour.

Each of the oligonucleotide probes preferably includes a GC content (molar percentage of guanine plus cytosine) of between about 40% to about 65%. More preferably, each of the oligonucleotide probes includes a GC content of between about 45% to about 60%. The design of an individual corresponding set of oligonucleotide probes is preferably such that each oligonucleotide probe of the set includes a sequence that permits a spacing of at least about 15 bases between adjacent oligonucleotide probes, when the adjacent oligonucleotide probes are hybridized to the corresponding target nucleic acid sequence.

Each of the oligonucleotide probes can include any type of nucleic acid, for example deoxyribonucleic acid (DNA), a ribonucleic acid (RNA), a nucleic acid mimic (such as, but not limited to, a peptide nucleic acid), or a combination thereof. Adjacent bases or nucleotides of the oligonucleotide probes may be joined by a bond other than a phosphodiester bond (for example, adjacent modifided bases or modified nucleotides may be joined by an amide bond, a phosphonate bond, a phosphorothioate bond, phosphorodithionate bond, a phosphoroamidite bond, a phosphate ester bond, a siloxane bond, a carbonate bond, an ester bond, a thioester bond, an acetamide bond, a carbamate bond, an acrylamide bond, an ethyleneimine bond, an ether bond, a thioether bond, or a boron-containing bond such as a P-boranomethylphosphonate bond), as is known in the art (see, for example, Hamma and Miller (2003) Antisense Nucleic Acid Drug Dev., 13: 19-30; Greenberg and Kahl (2001) J. Org. Chem., 66: 7151-7154; Lin and Shaw (2001) Nucleosides Nucleotides Nucleic Acids, 20: 1325-1328; Freier and Altmann (1997), Nucleic Acids Res., 25: 4429-4443; Rice and Gao (1997) Biochemistry, 36: 399-411; Agrawal et al. (1990), Proc. Natl. Acad. Sci. USA, 87: 1401-1405; and Shabarova (1988), Biochimie, 70: 1323-1334, which are incorporated by reference in their entirety herein). Nucleic acid mimics are artificial molecules that are structurally and functionally analogous to naturally occurring nucleic acids (deoxyribonucleic acids and ribonucleic acids). Nucleic acid mimics used in the method of the invention include bases that are analogous to the nucleotides found in naturally occurring nucleic acids, and that are capable of complementary base-pairing with the nucleotides in a naturally occurring nucleic acid. Non-limiting examples of a nucleic acid mimic include a nuclease-resistant boron-modified nucleotide polymer (Porter et al. (1997) Nucleic Acids Res., 25: 1611-1617), and a peptide nucleic acid (PNA), which contains purine and pyrimidine bases, and which has an aminoethylglycine backbone in place of the sugar-phosphate backbone of a naturally occurring nucleic acid (see, for example, Ganesh and Nielsen (2000) Curr. Org. Chem., 4: 931-943; Ray and Nordén (2000) FASEB J., 14: 1041-1060; Egholm et al. (1992) J. Am. Chem Soc., 114: 1895-1897, which are incorporated by reference in their entirety herein).

The oligonucleotide probes of the invention may be made by any technique suitable to the composition of the particular oligonucleotide probe. For example, an oligonucleotide probe may include only a nucleic acid (DNA or RNA) or only a nucleic acid mimic, and such an oligonucleotide probe may be made by any suitable DNA, RNA, or nucleic acid mimic synthesis method. See, generally, J; Sambrook and D. Russell, “Molecular Cloning: A Laboratory Manual”, third edition (2001), Cold Spring Harbor Laboratory Press, New York, 2,344 pp.; Braasch and Corey (2001) Methods, 23: 97-107; Hyrup and Nielsen (1996) Bioorg. Med. Chem., 4: 5-23; Sprout (1993) Curr. Opin. Biotechnol., 4: 20-28; and Gait (1991) Curr. Opin. Biotechnol., 2: 61-68, which are incorporated by reference in their entirety herein. The oligonucleotide probes may be hybrids or chimeras, preferably including a nucleic acid (DNA or RNA or both) or a nucleic acid mimic (such as, but not limited to, a peptide nucleic acid) or both; the oligonucleotide probes may further include a polypeptide, a polymer (such as polymeric plastics, silicones, fluorocarbons, polysaccharides, and the like), or a combination thereof. Oligonucleotide probes that are hybrids or chimeras may be manufactured by a combination of methods, including synthetic, semi-synthetic, enzymatic, recombinant, biological, or a combination thereof. See, generally, U.S. Pat. No. 6,204,326, issued 20 Mar. 2001, to Cook et al.; U.S. Pat. No. 5,539,083, issued 23 Jul. 1996, to Cook et al.; Tian and Wickstrom (2002) Org. Lett., 4: 4013-4016; Niemeyer (2002) Trends Biotechnol., 20: 395-401; Beier and Hoheisel (1999) Nucleic Acids Res., 27: 1970-1977; Efimov et al. (1999) Nucleic Acids Res., 27: 4416-4426; Koppitz et al. (1998) J. Am. Chem. Soc., 120: 4563-4569; and Misra et al. (1998) Biochemistry, 37: 1917-1925, which are incorporated by reference in their entirety herein.

The at least one detectable label of each of the oligonucleotide probes may be any suitable detectable label. Detectable labels include, but are not limited to, detectable nuclei (including radioactive isotopes such as tritium, radiocarbon, ³⁵S, ³²P, or ³³P, and non-radioactive isotopes), fluorophores, luminophores, dyes, pigments, members of resonance energy transfer pairs, spin labels, lanthanides, magnetic labels, detectable nucleic acids, metals, particles (such as, but not limited to, beads, fibers, or particles made of gold or other metals, magnetic or paramagnetic substances, glass, silicates, ceramics, latex, polymers, or composites), enzymes (such as peroxidase or alkaline phosphatase), antigenically recognizable structures (for example, digoxin or digoxigenin), and bindable moieties (for example, antibodies, antibody fragments, receptors, ligands, polyhistidine tags, biotin, or avidin). The at least one detectable label of each of the oligonucleotide probes may be detectable directly (for example, as is the case for oligonucleotide probes labelled with radioactive isotopes or fluorophores) or indirectly (for example, as is the case for oligonucleotide probes labelled with biotin and indirectly detected with avidin conjugated to a reporting enzyme or fluorophore).

Generally, the at least one detectable label is preferably the same between the corresponding sets of oligonucleotide probes, especially where the method of the invention is intended for quantitation of the at least two target nucleic acid sequences. In a given corresponding set of oligonucleotide probes, the at least one detectable label is most preferably identical for all the probes in that set. In some embodiments, the at least one detectable label may vary between each corresponding set of oligonucleotide probes; for example, one corresponding set of oligonucleotide probes may include a fluorophore label that is different from the fluorophore label of another corresponding set of oligonucleotide probes.

In one non-limiting embodiment of the invention, the detectable label can be incorporated during probe synthesis, for example, by incorporation of radioactively labelled bases such as ³⁵S-dNTP, ³²P-dNTP, ³³P-dNTP, ¹⁴C-dNTP, or by incorporation of non-radioactively labelled bases such as, but not limited to, digoxin- or digoxygenin-labelled dNTP, biotin-labelled dNTP, fluorophore-labelled dNTP, or hapten-labelled dNTP. In another example, the oligonucleotide is first synthesized from unlabelled bases (or, alternatively, with bases bearing functional groups to which a detectable label can be later attached), and the detectable label is attached to the probe, directly or indirectly, by covalent or non-covalent means or a combination thereof, after the oligonucleotide is synthesized. In some embodiments, the detectable label may be attached to the probe after hybridization occurs. For example, a biotinylated probe may hybridize to its target nucleic acid sequence, and be detected using an avidin-labelled enzyme and an appropriate enzyme substrate. Methods to introduce such functional groups or detectable labels are known in the art (see, for example, R. P. Haugland, “Handbook of Fluorescent Probes and Research Products”, 9^{th} edition, J. Gregory (editor), Molecular Probes, Inc., Eugene, Oreg., USA, 2002, 966 pp.; Seitz and Kohler (2001), Chemistry, 7: 3911-3925; Pierce Technical Handbook, Pierce Biotechnology, Inc., 1994, Rockford, Ill.; and Pierce 2003-2004 Applications Handbook and Catalog, Pierce Biotechnology, Inc., 2003, Rockford, Ill., which are incorporated by reference in their entirety herein). Where desired, for example, when increased flexibility is needed, a detectable label may be affixed to the oligonucleotide probe using a spacer arm (see for example, Keyes et al. (1997) Biophys. J, 72: 282-90; Hustedt et al. (1995) Biochemistry, 34: 4369-4375; and Pierce Technical Handbook, Pierce Biotechnology, Inc., 1994, Rockford, Ill., which are incorporated by reference in their entirety herein).

The method of the invention further includes the step of contacting the at least one sample with the corresponding sets of oligonucleotide probes so that each of the at least two target nucleic acid sequences is contacted with the corresponding set of oligonucleotide probes. By contacting is meant bringing the at least one sample suspected of containing the at least two target nucleic acid sequences in fluid contact, preferably in liquid contact, with the corresponding sets of oligonucleotide probes. Contacting the at least one sample with the corresponding sets of oligonucleotide probes may be carried out in a simultaneous or in a sequential manner. In one embodiment, all of the at least two target nucleic acid sequences are contacted simultaneously or substantially simultaneously with all of the corresponding sets of oligonucleotide probes. For example, the at least one sample suspected of containing two target nucleic acid sequences can be contacted with corresponding sets of oligonucleotide probes (where each set is preferably distinctively labelled, for example, labelled with a different fluorescent label) in a single contacting step, allowing each of the corresponding sets of probes to hybridise to their respective target nucleic acid sequence. In an alternative embodiment, the different corresponding sets of oligonucleotide probes are contacted sequentially with the at least one sample. For example, the at least one sample may be at least one membrane or at least one tissue section that can be contacted first with one corresponding set of oligonucleotide probes, which are allowed to hybridize to their target nucleic acid sequence and are detected; the at least one sample is then stripped of the first corresponding set of oligonucleotide probes, and the process repeated with the second and any subsequent corresponding sets of oligonucleotide probes.

In some embodiments, for example, where the detection is preferably quantitative, the at least one sample includes multiple samples (such as replicate or parallel or consecutive samples), which may be similar or substantially identical. Such multiple samples can be particularly useful in permitting parallel, optionally simultaneous, contacting, thus minimizing loss of target nucleic acid sequences, as can happen when a single sample is sequentially contacted with more than one set of oligonucleotide probes. Non-limiting examples of such multiple samples can include replicate membranes blots, replicate solution samples, microscopy sections taken in parallel fashion from separate tissues or organs, or microscopy sections taken in series from a tissue or organ. In certain cases, multiple samples are most preferred, for example, where in situ hybridization is carried out with sets of radioactively labelled oligonucleotide probes and multiple samples such as separate microscopy sections, optionally located on separate slides or areas of slides, or in separate wells or other containers.

Regardless of whether the contacting step is carried out in a simultaneous or in a sequential manner, all of the individual probes of a given corresponding set of oligonucleotide probes are most preferably contacted concurrently with the at least one sample, thus allowing the individual probes of the set to hybridize at the same time with the target nucleic acid sequence, if present in the at least one sample.

One non-limiting example of contacting is by immersing at least one sample (such as at least one gel or membrane or mounted tissue section) suspected of containing the at least two target nucleic acid sequences in a solution or solutions containing the corresponding sets of oligonucleotide probes. Another example is dispensing by pipette or other delivery means a volume of solution or solutions containing the corresponding sets of oligonucleotide probes onto discrete spots on a glass slide, wherein each spot contains at least one sample suspected of containing the at least two target nucleic acid sequences, affixed to the surface of the slide. Another example is in situ intracellular delivery of the corresponding sets of oligonucleotide probes, for example, of corresponding sets of oligonucleotide probes in a liquid solution, or in a suspension of liposomes, micelles, or lipid complexes (see, for example, Byk et al. (1998) J. Med. Chem., 41: 229-235; Fraley et al. (1981) Biochemistry, 20: 6978-6987, which are incorporated by reference in their entirety herein), to at least one sample including a whole cell or intact tissue.

The method of the invention further includes the step of incubating the at least one sample and the corresponding sets of oligonucleotide probes under conditions that allow hybridization of each of the target nucleic acid sequences, if present in the at least one sample, to the corresponding set of oligonucleotide probes. The at least one sample is incubated with the corresponding sets of oligonucleotide probes under hybridizing conditions for a period of time sufficient to permit hybridization between each of the corresponding sets of oligonucleotide probes and its respective target nucleic acid sequence, if the target nucleic acid sequence is present in the at least one sample. By hybridization is meant complementary base-pairing between a sequence of bases on a first nucleic acid (or nucleic acid mimic) strand and a sequence of bases on a second nucleic acid (or nucleic acid mimic) strand. Preferably, hybridization occurs between the target nucleic acid sequence, if present in the at least one sample, and each oligonucleotide probe of the respective corresponding set of oligonucleotide probes. Preferably, hybridization conditions are selected to achieve significant hybridization between the corresponding sets of oligonucleotide probes and the at least two target nucleic acid sequences, if present in the at least one sample. Most preferably, hybridization conditions are selected to achieve quantitative or near-quantitative hybridization between the corresponding sets of oligonucleotide probes and the at least two target nucleic acid sequences, if present in the at least one sample.

Hybridization is dependent on factors known in the art (see for example, pp. 33-37 in “Nonradioactive In Situ Hybridization Application Manual”, Roche Applied Science, 2002, Indianapolis, Ind., which is incorporated by reference in its entirety herein), including, but not limited to, the length and specific sequence of the base sequences between which complementary base-pairing occurs; the effective concentrations of the corresponding sets of oligonucleotide probes and the at least two target nucleic acid sequences, if present in the at least one sample; the melting or annealing temperatures of the hybridization mixture; the nature of the solvent; and the amount of any components (for example, inorganic ions, especially monovalent or divalent cations, or organic solutes such as formamide or dextran sulfate, included in the solvent). Certain factors may be more easily or more conveniently controlled, such as the melting or annealing temperatures or the ionic strength of the hybridization mixture. Manipulation of hybridization conditions is routine for one versed in the art.

The period of time of incubation is preferably sufficient to permit significant hybridization between the corresponding sets of oligonucleotide probes and the at least two target nucleic acid sequences, if present in the at least one sample, and most preferably sufficient to permit quantitative or near-quantitative hybridization between corresponding sets of oligonucleotide probes and the at least two target nucleic acid sequences. The period of time also depends on the nature of the at least one sample. For example, a sample suspected of containing the at least two target nucleic acid sequences, and that consists of highly purified and concentrated DNA in solution, may require only a short hybridization time (such as from between about 1 second to about 1 minute or between about 1 second and about 10 minutes), whereas a sample suspected of containing the at least two target nucleic acid sequences, and that includes a cell or a tissue may require an extended hybridization time (such as from about 4 hours to overnight or about 24 hours or even longer). For convenience, the period of time of incubation is most preferably the shortest period of time that permits an amount of hybridization between the corresponding sets of oligonucleotide probes and the at least two target nucleic acid sequences, if present in the at least one sample, that is satisfactory for a specific purpose (in particular, that satisfies the quantitation requirements of the user). The preferred concentration of the reactants (in particular, of the corresponding sets of oligonucleotide probes), is one that allows a detectable signal, under the hybridization conditions selected for that particular combination, that gives an acceptable signal-to-noise (that is to say, the amount of signal due to the specific assay response divided by the background signal) ratio for the particular instrument or means of detecting the signal. Preferably, the concentration of the reactants is also chosen to minimize costs.

The method of the invention further includes the step of detecting the hybridization, wherein the detecting of the hybridization indicates the relative amounts of each of the at least two target nucleic acid sequences in the at least one sample. Detecting the hybridization may be by any means suitable to the type of signal or signals produced by the hybridization. Types of signals useful in the method of the invention include, but are not limited to, radioactivity, luminescence, chemiluminescence, fluorescence, light, color or wavelength change, production of a chemical or enzymatic product, and the like. In certain instances, detection may be of the absence of signal, wherein such absence is indicative of hybridization (for example, where the probes include molecular beacons).

Detection of hybridization in certain types of samples, such as in membrane blots or in situ in microscopy mounts, may be, for example, by colorimetry or by autoradiography using photographic film, emulsion, or other media, optionally including densitometric analysis or other quantitation as is known in the art. See, for example, “Nonradioactive In Situ Hybridization Application Manual”, Roche Applied Science, 2002, Indianapolis, Ind., which is incorporated by reference in its entirety herein. Detection may optionally make use of one or more detectable binding molecules (such as a detectable antibody or antibody fragment, receptor, hapten, or ligand), a labelling or conversion reaction that results in a detectable signal (for example, a chemical, photochemical, or physical treatment of a precursor to a fluorophore that results in a fluorescent signal), or an enzymatic or amplification reaction. Other suitable detection means include densitometers, phosphorimagers, calorimeters, spectrophotometers, fluorimeters, luminometers, nuclear magnetic resonance (NMR) spectrometers, electron spin resonance (ESR) spectrometers, electron paramagnetic resonance (EPR) spectrometers, cameras, charge-coupled detectors, photodiodes, photodiode arrays, photomultipliers, or other light sensors with filters or wavelength selection filters or devices, light microscopes, fluorescence microscopes, epifluorescence microscopes, confocal microscopes, electron microscopes, near field scanning optical microscopes, far field confocal microscopes, scanning probe microscopes (such as scanning tunneling microscopes and atomic force microscopes), or a combination of these. The detection means may be adapted to detect a signal in different assay formats, for example, single-use chambers (such as tubes or cuvettes), flow-through chambers, microtiter plates, microarrays, spots on a hybridization slide or chip, mounted cell or tissue sections, beads, optical fibers, and the like. The detection means may form part of a larger apparatus (which may be suited to high-throughput screening), such as a microplate reader, a liquid chromatograph, an electrophoretic capillary apparatus, a sheath-flow apparatus (such as a flow cytometer), or a video apparatus. Some embodiments may also use computerized methods to detect or amplify detection of the complex, for example, using computers to integrate a signal over time, to interpolate between known data points, or to increase signal-to-noise ratios. In some embodiments, detection may be made visually, without magnification or other signal amplification.

Detecting of the hybridization preferably indicates the relative amounts of each of the at least two target nucleic acid sequences in the at least one sample. Detection may be quantitative, semi-quantitative, or qualitative in nature. Detection can be linear (such as densitometric measurement of a photographic film, or spectrophotometric measurement of product formation by an enzymatic reaction) or non-linear (such as visual detection of a gold label or a precipitated enzymatic substrate). By “relative” is meant that the detected amounts of each of the at least two target nucleic acid sequences in the at least one sample can be compared to each other, or to a reference value or values, or to separately measured (in a spatial or temporal sense) amounts of the same or different target nucleic acid sequences. For example, the amounts of each of the at least two target nucleic acid sequences in the at least one sample can be compared to amounts of the same or different target nucleic acid sequences detected in previous or succeeding experiments, or a sample previously analysed can be reanalysed with new sets of oligonucleotide probes. In one embodiment, the amounts of each of the at least two target nucleic acid sequences can be compared to a reference value or values, which may be calculated or obtained by empirical measurements. In another embodiment, where detection of the at least two target nucleic acid sequences is intended to be qualitative, the detected amount of each of the at least two target nucleic acid sequences may be simply considered “positive” if it is greater than or equal to a reference value, or “negative” if it is less than a reference value. In yet other embodiments, detection of the at least two target nucleic acid sequences may be semi-quantitative or quantitative, where the detected amounts can be correlated to ranges of values or to more precise values. Standards (such as, but not limited to, standards for specific radioactivity, optical density, or enzymatic activity) may be used in order to obtain such ranges of values or more precise values. In one non-limiting embodiment, when the signal to be detected is radioactivity or light detected on film or by other sensing means, longer exposures can be used to increase the signal, preferably maintaining the signal obtained within the linear range of the standards. Comparison of the amounts of each of the at least two target nucleic acid sequences most preferably takes into consideration any factors that may affect accurate quantification of the amounts, such as, but not limited to, any known variability in oligonucleotide probe length (X) or numbers (N), and differences in detectable label characteristics (for example, specific activity of a radioactive isotope label, or different fluorescence excitation and emission wavelengths and extinction coefficients).

II. Method to Detect at Least Two Target Messenger RNAs

The present invention also includes a method to detect the relative amounts of at least two target messenger RNAs (mRNAs) in at least one microscopy sample, including providing at least one microscopy sample suspected of containing the at least two target messenger RNAs; providing a corresponding set of oligonucleotide probes for each of the at least two target messenger RNAs, wherein each of the corresponding sets of oligonucleotide probes comprises N oligonucleotide probes, where N comprises between about 2 to about 24 oligonucleotide probes, and wherein each of the oligonucleotide probes comprises a sequence comprising X bases, where X comprises between about 15 to about 60 bases, and comprising a GC content of between about 40% to about 65%, and permitting a spacing of at least about 15 bases between adjacent oligonucleotide probes when the adjacent oligonucleotide probes are hybridized to the corresponding the target messenger RNA; and at least one detectable label; contacting the at least one microscopy sample with the corresponding sets of oligonucleotide probes so that each of the at least two target messenger RNAs is contacted with the corresponding set of oligonucleotide probes; incubating the at least one microscopy sample and the corresponding sets of oligonucleotide probes under conditions that allow in situ hybridization of each of the target messenger RNAs, if present in the at least one microscopy sample, to the corresponding set of oligonucleotide probes; and detecting said in situ hybridization, wherein the detecting of said in situ hybridization indicates the relative amounts of each of the at least two target messenger RNAs in the at least one microscopy sample.

The method of the present invention may be applied to any at least one suitable microscopy sample that is suspected of containing the at least two target messenger RNAs of interest. Suitable microscopy samples can include whole cells or cell fragments or cell components, tissues, organs, or biological fluids, or cultured cells. Tissues can include any one or more tissues of interest (for example, neural, muscular, epithelial, and connective tissues). Suitable microscopy samples can include one or more whole organs or part of organs or anatomic structures (for example, brains, livers, heart, skin, bones, secretory organs, and reproductive organs). The at least one microscopy sample may be treated as necessary or desirable to improve its characteristics for microscopic purposes, for example, by dissection, fixation, embedding, sectioning, staining, or other preparation for microscopy. The at least one microscopy sample may be subjected to other preparation steps, such as, but not limited to, heating or cooling; removal, inactivation, or blocking of undesirable material, such as contaminants, undesired nucleic acids, or endogenous enzymes; treatment with chemicals, enzymes, permeabilizing agents, or physical treatments. The at least one microscopy sample may be subjected to nucleic acid amplification reactions, such as, but not limited to, reverse transcriptase amplification of RNA. Examples of in situ amplification of a nucleic acid (Long (1998) Eur. J. Histochem., 42: 101-109, which is incorporated by reference in its entirety herein), or a cellular structure that contains a nucleic acid (Lichter et al. (1990), Science, 247: 64-69, which is incorporated by reference in its entirety herein) are known in the art.

The method of the present invention may be applied to at least two target messenger RNAs of interest. In one embodiment of the method, the target messenger RNAs or RNA transcripts can be subjected to amplification, for example, to reverse transcriptase amplification.

The method of the present invention includes the step of providing a corresponding set of oligonucleotide probes for each of the at least two target messenger RNAs. Thus, a corresponding set of oligonucleotide probes is provided for each individual target messenger RNAs. In general, the corresponding sets of oligonucleotide probes are similar to those described above under the heading “I. METHOD TO DETECT AT LEAST Two TARGET NUCLEIC ACID SEQUENCES”. Each corresponding set of oligonucleotide probes includes N oligonucleotide probes, wherein N is an integer greater than 1. Preferably N ranges from about 2 to about 24, or from about 2 to about 14, or from about 2 to about 12 or from about 3 to about 10, or from about 4 to about 10. Preferably, the value of N is identical between corresponding sets of oligonucleotide probes. However, in some embodiments of the method, N may not be identical between corresponding sets of oligonucleotide probes, and the differing numbers of oligonucleotide probes in each corresponding set must be taken into account if accurate quantification of the amounts of each of the at least two target messenger RNAs is desired.

N, the number of oligonucleotide probes per set, is preferably selected according to the expected abundance or scarcity of the target messenger RNAs in the at least one microscopy sample. As a general practice, the less abundant the target messenger RNA is, the larger N preferably is. In one embodiment, the messenger RNAs can be predicted or expected to be characterized by “low copy number” (about 1 to about 30 copies per cell), “medium copy number” (about 30 to about 1000 copies per cell), or “high copy number” (greater than about 1000 copies to about 100,000 copies per cell). In this non-limiting embodiment, typical values of N for these ranges can be from about 3 to about 4 oligonucleotide probes for “high copy number” messenger RNAs, from about 5 to about 8 probes for “medium copy number” messenger RNAs, and from about 9 to about 12 probes for “low copy number” messenger RNAs. However, these values of N merely serve as guidelines, and the actual N may be varied by the user according to his or her requirements.

In general, the oligonucleotide probes are similar to those described above under the heading “I. METHOD TO DETECT AT LEAST Two TARGET NUCLEIC ACID SEQUENCES”. Each of the oligonucleotide probes is designed to include a sequence that is complementary to at least part of the corresponding target messenger RNA. Each of the oligonucleotide probes most preferably includes a sequence that is specifically complementary to at least part of the corresponding target messenger RNA. By “specifically complementary” is meant that the oligonucleotide probe's sequence is complementary to its intended target messenger RNA, and preferably does not show substantial similarity or homology to messenger RNAs or other nucleic acid sequences that are not of interest. Thus, each oligonucleotide sequence is preferably compared against known sequences, such as, but not limited to, sequences publicly available in the databases maintained by the National Center for Biotechnology Information (accessible on-line at www.ncbi.nlm.nih.gov), to confirm that the oligonucleotide sequence is specifically complementary to the target messenger RNA, and preferably does not show substantial similarity or homology to other known messenger RNAs or other nucleic acid sequences.

Each of the oligonucleotide probes includes a sequence of X bases (or nucleotides) and at least one detectable label, wherein X is an integer. Preferably X ranges from about 15 to about 60 bases, or from about 20 to about 50 bases, or about 20 to about 40 bases. In a given corresponding set of oligonucleotide probes, X is preferably identical for all the probes in order to minimize variability in probe length, probe GC content, probe annealing and melting temperatures, and other factors that influence hybridization behaviour. In some embodiments, particularly where the probes are longer probes, X may vary slightly between the probes of a given corresponding set of oligonucleotide probes, if such variation does not result in substantial unwanted variability in hybridization behaviour.

Each of the oligonucleotide probes preferably includes a GC content of between about 40% to about 65%. More preferably, each of the oligonucleotide probes includes a GC content of between about 45% to about 60%. The design of an individual corresponding set of oligonucleotide probes is preferably such that each oligonucleotide probe of the set includes a sequence that permits a spacing of at least about 15 bases between adjacent oligonucleotide probes, when the adjacent oligonucleotide probes are hybridized to the corresponding target messenger RNA.

Each of the oligonucleotide probes can include any type of nucleic acid, for example deoxyribonucleic acid (DNA), a ribonucleic acid (RNA), a nucleic acid mimic (such as, but not limited to, a peptide nucleic acid), or a combination thereof. Adjacent bases or nucleotides of the oligonucleotide probes may be joined by a bond other than a phosphodiester bond, as is known in the art.

The oligonucleotide probes of the invention may be made by any technique suitable to the composition of the particular oligonucleotide probe. For example, an oligonucleotide probe may include only a nucleic acid (DNA or RNA) or only a nucleic acid mimic, and such an oligonucleotide probe may be made by any suitable DNA, RNA, or nucleic acid mimic synthesis method. The oligonucleotide probes may be hybrids or chimeras, preferably including a nucleic acid (DNA or RNA or both) or a nucleic acid mimic (such as, but not limited to, a peptide nucleic acid) or both; the oligonucleotide probes may further include a polypeptide, a polymer, or a combination thereof. Oligonucleotide probes that are hybrids or chimeras may be manufactured by a combination of methods, including synthetic, semi-synthetic, enzymatic, recombinant, biological, or a combination thereof.

The at least one detectable label of each of the oligonucleotide probes may be any suitable detectable label. Detectable labels include, but are not limited to, detectable nuclei (including radioactive isotopes and non-radioactive isotopes), fluorophores, luminophores, dyes, pigments, members of resonance energy transfer pairs, spin labels, lanthanides, magnetic labels, detectable nucleic acids, metals, particles, enzymes, antigenically recognizable structures, and bindable moieties. The at least one detectable label of each of the oligonucleotide probes may be detectable directly or indirectly.

Generally, the at least one detectable label is preferably the same between the corresponding sets of oligonucleotide probes, especially where the method of the invention is intended for quantitation of the at least two target messenger RNAs. In a given corresponding set of oligonucleotide probes, the at least one detectable label is most preferably identical for all the probes in that set. In some embodiments, the at least one detectable label may vary between each corresponding set of oligonucleotide probes; for example, one corresponding set of oligonucleotide probes may include a fluorophore label that is different from the fluorophore label of another corresponding set of oligonucleotide probes. The detectable label can be incorporated during probe synthesis. Alternatively, the oligonucleotide is first synthesized from unlabelled bases (or, with bases bearing functional groups to which a detectable label can be later attached), and the detectable label is attached to the probe, directly or indirectly, by covalent or non-covalent means or a combination thereof, after the oligonucleotide is synthesized. In some embodiments, the detectable label may be attached to the probe after hybridization occurs.

The method of the invention further includes the step of contacting the at least one microscopy sample with the corresponding sets of oligonucleotide probes so that each of the at least two target messenger RNAs is contacted with the corresponding set of oligonucleotide probes. By contacting is meant bringing the at least one microscopy sample suspected of containing the at least two target messenger RNAs in fluid contact, preferably in liquid contact, with the corresponding sets of oligonucleotide probes. Contacting the at least one microscopy sample with the corresponding sets of oligonucleotide probes may be carried out in a simultaneous or in a sequential manner. For example, at least one microscopy section can be sequentially contacted, first with one corresponding set of oligonucleotide probes, which are allowed to hybridize to their target messenger RNA and are detected; the at least one microscopy section is then stripped of the first corresponding set of oligonucleotide probes, and the process repeated with the second and any subsequent corresponding sets of oligonucleotide probes.

In some embodiments, for example, where the detection is preferably quantitative, the at least one microscopy sample includes multiple microscopy samples (such as replicate or parallel or consecutive microscopy samples), which may be similar or substantially identical. Such multiple microscopy samples can be particularly useful in permitting simultaneous contacting, thus minimizing loss of target nucleic acid sequences, as can happen when a single microscopy sample is sequentially contacted with more than one set of oligonucleotide probes. Non-limiting examples of such multiple microscopy samples can include microscopy sections taken in parallel fashion from separate tissues or organs (for example, sections taken in parallel fashion from brains obtained from separate animals), or multiple microscopy sections taken in series from a tissue or organ. In certain cases, multiple microscopy samples are most preferred, for example, where in situ hybridization is carried out with sets of radioactively labelled oligonucleotide probes and separate microscopy sections, optionally located on separate slides or areas of slides, or in separate wells or other containers.

Regardless of whether the contacting step is carried out in a simultaneous or in a sequential manner, all of the individual probes of a given corresponding set of oligonucleotide probes are most preferably contacted concurrently with the microscopy sample, thus allowing the individual probes of the set to hybridize at the same time with the target messenger RNA, if present in the at least one microscopy sample. A non-limiting example of contacting is in situ intracellular delivery of the corresponding sets of oligonucleotide probes, for example, of corresponding sets of oligonucleotide probes in a liquid solution, or in a suspension of liposomes, micelles, or lipid complexes (Byk et al. (1998) J. Med. Chem., 41: 229-235; Fraley et al. (1981) Biochemistry, 20: 6978-6987), to at least one microscopy sample.

The method of the invention further includes the step of incubating the at least one microscopy sample and the corresponding sets of oligonucleotide probes under conditions that allow hybridization of each of the target messenger RNAs, if present in the at least one microscopy sample, to the corresponding set of oligonucleotide probes. The at least one microscopy sample is incubated with the corresponding sets of oligonucleotide probes under hybridizing conditions for a period of time sufficient to permit hybridization between each of the corresponding sets of oligonucleotide probes and its respective target messenger RNA, if the target messenger RNA is present in the at least one microscopy sample. By hybridization is meant complementary base-pairing between a sequence of bases on a first nucleic acid (or nucleic acid mimic) strand and a sequence of bases on a second nucleic acid (or nucleic acid mimic) strand. Preferably, hybridization occurs between the target messenger RNA, if present in the at least one microscopy sample, and each oligonucleotide probe of the respective corresponding set of oligonucleotide probes. Preferably, hybridization conditions are selected to achieve significant hybridization between the corresponding sets of oligonucleotide probes and the at least two target messenger RNAs, if present in the at least one microscopy sample. Most preferably, hybridization conditions are selected to achieve quantitative or near-quantitative hybridization between the corresponding sets of oligonucleotide probes and the at least two target messenger RNAs, if present in the microscopy sample. Hybridization is dependent on factors known in the art (see for example, pp. 33-37 in “Nonradioactive In Situ Hybridization Application Manual”, Roche Applied Science, 2002, Indianapolis, Ind., which is incorporated by reference in its entirety herein), and manipulation of hybridization conditions would be routine for one versed in the art.

The period of time of incubation is preferably sufficient to permit significant hybridization between the corresponding sets of oligonucleotide probes and the at least two target messenger RNAs, if present in the at least one microscopy sample, and most preferably sufficient to permit quantitative or near-quantitative hybridization between corresponding sets of oligonucleotide probes and the at least two target messenger RNAs. The period of time also depends on the nature of the at least one microscopy sample (for example, on the at least one microscopy sample's thickness or density or degree of permeabilization). For convenience, the period of time of incubation is most preferably the shortest period of time that permits an amount of hybridization between the corresponding sets of oligonucleotide probes and the at least two target messenger RNAs, if present in the at least one microscopy sample, that is satisfactory for a specific purpose (in particular, that satisfies the need for quantitation requirements of the user). The preferred concentration of the reactants (in particular, of the corresponding sets of oligonucleotide probes), is one that allows a detectable signal, under the hybridization conditions selected for that particular combination, that gives an acceptable signal-to-noise (that is to say, the amount of signal due to the specific assay response divided by the background signal) ratio for the particular instrument or means of detecting the signal. Preferably, the concentration of the reactants is also chosen to minimize costs.

The method of the invention further includes the step of detecting the hybridization, wherein the detecting of the hybridization indicates the relative amounts of each of the at least two target messenger RNAs in the at least one microscopy sample. Detecting the hybridization may be by any means suitable to the type of signal or signals produced by the hybridization. Types of signals useful in the method of the invention include, but are not limited to, radioactivity, luminescence, chemiluminescence, fluorescence, light, color or wavelength change, production of a chemical or enzymatic product, and the like. In certain instances, detection may be of the absence of signal, wherein such absence is indicative of hybridization (for example, where the probes include molecular beacons).

Detection of in situ hybridization in the microscopy sample, may be, for example, by colorimetry or by autoradiography using photographic film, emulsion, or other media, optionally including densitometric analysis or other quantitation as is known in the art. See, for example, “Nonradioactive In Situ Hybridization Application Manual”, Roche Applied Science, 2002, Indianapolis, Ind., which is incorporated by reference in its entirety herein. Detection may optionally make use of one or more detectable binding molecules, a labelling or conversion reaction that results in a detectable signal, or an enzymatic or amplification reaction. Other suitable detection means include, but are not limited to, densitometers, phosphorimagers, calorimeters, cameras, charge-coupled detectors, photodiodes, photodiode arrays, photomultipliers, or other light sensors with filters or wavelength selection filters or devices, light microscopes, fluorescence microscopes, epifluorescence microscopes, confocal microscopes, electron microscopes, near field scanning optical microscopes, far field confocal microscopes, scanning probe microscopes (such as scanning tunneling microscopes and atomic force microscopes), or a combination of these. The detection means may be adapted to detect a signal in different assay formats, and may form part of a larger apparatus (which may be suited to high-throughput screening). Some embodiments may also use computerized methods to detect or amplify detection of the complex. In some embodiments, detection may be made visually, without magnification or other signal amplification.

Detecting of the in situ hybridization preferably indicates the relative amounts of each of the at least two target messenger RNAs in the at least one microscopy sample. Detection may be quantitative, semi-quantitative, or qualitative in nature. Detection can be linear or non-linear. By “relative” is meant that the detected amounts of each of the at least two target messenger RNAs in the sample can be compared to each other, or to a reference value or values, or to separately measured (in a spatial or temporal sense) amounts of the same or different target messenger RNAs. Standards may be used in order to obtain ranges of values or more precise values to which the detected amounts of the at least two target messenger RNAs may be compared. When the signal to be detected is radioactivity or light detected on film or by other sensing means, longer exposures can be used to increase the signal, preferably maintaining the signal obtained within the linear range of the standards. Comparison of the amounts of each of the at least two target messenger RNAs most preferably takes into consideration any factors that may affect accurate quantification of the amounts.

III. Method to Provide Sets of Oligonucleotide Probes

The present invention also includes a method to provide sets of oligonucleotide probes useful in detecting at least two target nucleic acid sequences, including the steps of: (a) selecting said at least two target nucleic acid sequences; (b) designing a corresponding set of oligonucleotide probes for each of the at least two target nucleic acid sequences, wherein each of the corresponding sets of oligonucleotide probes includes N oligonucleotide probes, where N includes between about 2 to about 24 oligonucleotide probes, and wherein each of the oligonucleotide probes includes (i) a sequence specifically complementary to at least part of the corresponding target nucleic acid sequence, and including X bases, where X includes between about 15 to about 60 bases, and including a GC content of between about 40% to about 65%, and permitting a spacing of at least about 15 bases between adjacent oligonucleotide probes when the adjacent oligonucleotide probes are hybridized to the corresponding target nucleic acid sequence; and (ii) at least one detectable label; and (c) synthesizing the designed corresponding sets of oligonucleotide probes for each of the at least two target nucleic acid sequences. The method of the present invention preferably provides sets of oligonucleotide probes useful in detecting at least two target nucleic acid sequences, as described above under the headings “I. METHOD TO DETECT AT LEAST Two TARGET NUCLEIC ACID SEQUENCES” and “II. METHOD TO DETECT AT LEAST Two TARGET MESSENGER RNAS”.

Any at least two target nucleic acid sequences of interest may be selected for use in the method of the invention. Suitable target nucleic acid sequences can include any type of nucleic acid, for example, DNA or RNA, or a nucleic acid mimic (such as, but not limited to, a peptide nucleic acid), or a combination thereof. In one embodiment of the method, the target nucleic acid sequences can include messenger RNAs or RNA transcripts. In another embodiment of the method, the target nucleic acid sequences can include genomic DNA. In yet another embodiment of the method, the target nucleic acid sequences can include nucleic acid constructs such as plasmids optionally including inserted sequences.

The method of the present invention includes the step of designing a corresponding set of oligonucleotide probes for each of the at least two target nucleic acid sequences. Thus, a corresponding set of oligonucleotide probes is designed for each individual target nucleic acid sequence. Each of the oligonucleotide probes can include any type of nucleic acid, for example deoxyribonucleic acid (DNA), a ribonucleic acid (RNA), a nucleic acid mimic (such as, but not limited to, a peptide nucleic acid), or a combination thereof. Adjacent bases or nucleotides of the oligonucleotide probes may be joined by a bond other than a phosphodiester bond (for example, adjacent modifided bases or modified nucleotides may be joined by an amide bond, a phosphonate bond, a phosphorothioate bond, phosphorodithionate bond, a phosphoroamidite bond, a phosphate ester bond, a siloxane bond, a carbonate bond, an ester bond, a thioester bond, an acetamide bond, a carbamate bond, an acrylamide bond, an ethyleneimine bond, an ether bond, a thioether bond, or a boron-containing bond such as a P-boranomethylphosphonate bond), as is known in the art (see, for example, Hamma and Miller (2003) Antisense Nucleic Acid Drug Dev., 13: 19-30; Greenberg and Kahl (2001) J. Org. Chem., 66: 7151-7154; Lin and Shaw (2001) Nucleosides Nucleotides Nucleic Acids, 20: 1325-1328; Freier and Altmann (1997), Nucleic Acids Res., 25: 4429-4443; Rice and Gao (1997) Biochemistry, 36: 399-411; Agrawal et al. (1990), Proc. Natl. Acad. Sci. USA, 87: 1401-1405; and Shabarova (1988), Biochimie, 70: 1323-1334, which are incorporated by reference in their entirety herein). Nucleic acid mimics are artificial molecules that are structurally and functionally analogous to naturally occurring nucleic acids (deoxyribonucleic acids and ribonucleic acids). Nucleic acid mimics used in the method of the invention include bases that are analogous to the nucleotides found in naturally occurring nucleic acids, and that are capable of complementary base-pairing with the nucleotides in a naturally occurring nucleic acid. Non-limiting examples of a nucleic acid mimic include a nuclease-resistant boron-modified nucleotide polymer (Porter et al. (1997) Nucleic Acids Res., 25: 1611-1617), and a peptide nucleic acid (PNA), which contains purine and pyrimidine bases, and which has an aminoethylglycine backbone in place of the sugar-phosphate backbone of a naturally occurring nucleic acid (see, for example, Ganesh and Nielsen (2000) Curr. Org. Chem., 4: 931-943; Ray and Nordén (2000) FASEB J, 14: 1041-1060; Egholm et al. (1992) J. Am. Chem Soc., 114: 1895-1897, which are incorporated by reference in their entirety herein).

Each corresponding set of oligonucleotide probes is designed to include N oligonucleotide probes, wherein N is an integer greater than 1. Preferably N ranges from about 2 to about 24, or from about 2 to about 20, or from about 2 to about 14, or from about 2 to about 12 or from about 3 to about 10, or from about 4 to about 10. Preferably, the value of N is identical between corresponding sets of oligonucleotide probes. In some embodiments of the method, N may not be identical between corresponding sets of oligonucleotide probes, and the differing numbers of oligonucleotide probes in each corresponding set must be taken into account if accurate quantification of the amounts of each of the at least two target nucleic acid sequences is desired.

N, the number of oligonucleotide probes per set, is preferably selected according to the expected abundance or scarcity of the target nucleic acid sequence in the sample. As a general practice, the less abundant the target nucleic acid sequence is, the larger N should be. In one embodiment where the target nucleic acid sequences are messenger RNAs, the messenger RNAs can be predicted or expected to be characterized by “low copy number” (about 1 to about 30 copies per cell), “medium copy number” (about 30 to about 1000 copies per cell), or “high copy number” (greater than about 1000 copies to about 100,000 copies per cell). In this non-limiting embodiment, typical values of N for these ranges can be from about 3 to about 4 oligonucleotide probes for “high copy number” messenger RNAs, from about 5 to about 8 probes for “medium copy number” messenger RNAs, and from about 9 to about 12 probes for “low copy number” messenger RNAs. However, these values of N merely serve as guidelines for this embodiment, and the actual N may be varied by the user according to his or her requirements.

Each of the oligonucleotide probes is designed to include a sequence that is complementary to at least part of the corresponding target nucleic acid sequence. By “complementary” is meant that stable hydrogen bonding occurs between a purine base and a pyrimidine base according to Watson-Crick base-pairing rules, such as is seen in double-stranded naturally occurring nucleic acids where the pair of bases consists of a purine base (adenine or guanine) on one strand of nucleic acid and a pyrimidine base (thymine, cytosine, or uracil) on a second and opposite-running strand of nucleic acid. According to Watson-Crick base-pairing rules, adenine base-pairs with thymine (in deoxyribonucleic acids) or with uracil (in ribonucleic acids), and guanine base-pairs with cytosine. Analogous complementary base-pairing may also occur between bases of a nucleic acid mimic (such as, but not limited to, a peptide nucleic acid) and nucleotides of a naturally occurring nucleic acid. By “specifically complementary” is meant that the oligonucleotide probe's sequence is complementary to its intended target nucleic acid sequence, and preferably does not show substantial similarity or homology to nucleic acid sequences that are not of interest. In some embodiments, the oligonucleotide probe's sequence is preferably at least 90% complementary, and more preferably at least 95% complementary, to its intended target nucleic acid sequence. In some embodiments, the oligonucleotide probe is most preferably 100% complementary to its intended target nucleic acid. Thus, each oligonucleotide sequence is preferably compared against known sequences, such as, but not limited to, sequences publicly available in the databases maintained by the National Center for Biotechnology Information (accessible on-line at www.ncbi.nlm.nih.gov), to confirm that the oligonucleotide sequence is specifically complementary to the target nucleic acid sequence, and preferably does not show substantial similarity or homology to other known nucleic acid sequences.

Each of the oligonucleotide probes is designed to include X bases (or nucleotides), wherein X is an integer. Preferably X ranges from about 15 to about 60 bases, or from about 20 to about 50 bases, or about 20 to about 40 bases. In a given corresponding set of oligonucleotide probes, X is preferably identical for all the probes in order to minimize variability in probe length, probe GC content, probe annealing and melting temperatures, and other factors that influence hybridization behaviour. In some embodiments, particularly where the probes are longer probes, X may vary slightly between the probes of a given corresponding set of oligonucleotide probes, if such variation does not result in substantial unwanted variability in hybridization behaviour.

Each of the oligonucleotide probes is designed to include a GC content (molar percentage of guanine plus cytosine) preferably of between about 40% to about 65%. More preferably, each of the oligonucleotide probes is designed to include a GC content of between about 45% to about 60%. Thus, when selecting the complementary sequence of each of the oligonucleotide probes, it is preferable to select a sequence complementary to a region in the target nucleic acid sequence that is neither GC-rich nor AT-rich. In some embodiments of the invention, it is also preferable to avoid sequences that include more than five identical nucleotides in series.

Each of the oligonucleotide probes is designed to permit a spacing of preferably at least about 15 bases between adjacent oligonucleotide probes when the adjacent oligonucleotide probes are hybridized to the corresponding target nucleic acid sequence.

Each of the oligonucleotide-probes is designed to include at least one detectable label. The at least one detectable label of each of the oligonucleotide probes may be any suitable detectable label. Detectable labels include, but are not limited to, detectable nuclei (including radioactive isotopes such as tritium, radiocarbon, ³⁵S, ³²P, or ³³P, and non-radioactive isotopes), fluorophores, luminophores, dyes, pigments, members of resonance energy transfer pairs, spin labels, lanthanides, magnetic labels, detectable nucleic acids, metals, particles (such as, but not limited to, beads, fibers, or particles made of gold or other metals, magnetic or paramagnetic substances, glass, silicates, ceramics, latex, polymers, or composites), enzymes (such as peroxidase or alkaline phosphatase), antigenically recognizable structures (for example, digoxin or digoxigenin), and bindable moieties (for example, antibodies, antibody fragments, receptors, ligands, polyhistidine tags, biotin, or avidin). The at least one detectable label of each of the oligonucleotide probes may be detectable directly (for example, as is the case for oligonucleotide probes labelled with radioactive isotopes or fluorophores) or indirectly (for example, as is the case for oligonucleotide probes labelled with biotin and indirectly detected with avidin conjugated to a reporting enzyme or fluorophore).

Generally, the at least one detectable label is preferably the same between the sets of oligonucleotide probes, especially where the sets of oligonucleotide probes are intended for use in quantitation of the at least two target nucleic acid sequences. In a given set of oligonucleotide probes, the at least one detectable label is most preferably identical for all the probes in that set. In some embodiments, the at least one detectable label may vary between each set of oligonucleotide probes; for example, one set of oligonucleotide probes may include a fluorophore label that is different from the fluorophore label of another corresponding set of oligonucleotide probes.

In one non-limiting embodiment of the invention, the detectable label can be incorporated during probe synthesis, for example, by incorporation of radioactively labelled bases such as ³⁵S-dNTP, ³²P-dNTP, ³³P-dNTP, ¹⁴C-dNTP, or by incorporation of non-radioactively labelled bases such as, but not limited to, digoxin- or digoxygenin-labelled dNTP, biotin-labelled dNTP, fluorophore-labelled dNTP, or hapten-labelled dNTP. In one non-limiting example, oligonucleotide probes intended for use in in situ hybridization can be internally labelled during synthesis using a modified amino-allyl-dT; in such a case it may be preferable to choose a sequence containing 3 adenines at relatively equal spacing intervals, which would allow the same oligonucleotide probe sequence to be used for both radioactive and non-radioactive in situ hybridization. In another example, the oligonucleotide is first synthesized from unlabelled bases (or, alternatively, with bases bearing functional groups to which a detectable label can be later attached), and the detectable label is attached to the probe, directly or indirectly, by covalent or non-covalent means or a combination thereof, after the oligonucleotide is synthesized. In some embodiments, the detectable label may be attached to the probe after hybridization occurs. For example, a biotinylated probe may hybridize to its target nucleic acid sequence, and be detected using an avidin-labelled enzyme and an appropriate enzyme substrate. Methods to introduce such functional groups or detectable labels are known in the art (see, for example, R. P. Haugland, “Handbook of Fluorescent Probes and Research Products”, 9^{th} edition, J. Gregory (editor), Molecular Probes, Inc., Eugene, Oreg., USA, 2002, 966 pp.; Seitz and Kohler (2001), Chemistry, 7: 3911-3925; Pierce Technical Handbook, Pierce Biotechnology, Inc., 1994, Rockford, Ill.; and Pierce 2003-2004 Applications Handbook and Catalog, Pierce Biotechnology, Inc., 2003, Rockford, Ill., which are incorporated by reference in their entirety herein). Where desired, for example, when increased flexibility is needed, a detectable label may be affixed to the oligonucleotide probe using a spacer arm (see for example, Keyes et al. (1997) Biophys. J., 72: 282-90; Hustedt et al. (1995) Biochemistry, 34: 4369-4375; and Pierce Technical Handbook, Pierce Biotechnology, Inc., 1994, Rockford, Ill., which are incorporated by reference in their entirety herein).

The sets of oligonucleotide probes of the invention may be made by any technique suitable to the composition of the particular oligonucleotide probe. For example, an oligonucleotide probe may include only a nucleic acid (DNA or RNA) or only a nucleic acid mimic, and such an oligonucleotide probe may be made by any suitable DNA, RNA, or nucleic acid mimic synthesis method as is known in the art. See, generally, J. Sambrook and D. Russell, “Molecular Cloning: A Laboratory Manual”, third edition (2001), Cold Spring Harbor Laboratory Press, New York, 2,344 pp.; Braasch and Corey (2001) Methods, 23: 97-107; Hyrup and Nielsen (1996) Bioorg. Med. Chem., 4: 5-23; Sprout (1993) Curr. Opin. Biotechnol., 4: 20-28; and Gait (1991) Curr. Opin. Biotechnol., 2: 61-68, which are incorporated by reference in their entirety herein. The oligonucleotide probes may be hybrids or chimeras, preferably including a nucleic acid (DNA or RNA or both) or a nucleic acid mimic (such as, but not limited to, a peptide nucleic acid) or both; the oligonucleotide probes may further include a polypeptide, a polymer (such as polymeric plastics, silicones, fluorocarbons, polysaccharides, and the like), or a combination thereof. Oligonucleotide probes that are hybrids or chimeras may be manufactured by a combination of methods, including synthetic, semi-synthetic, enzymatic, recombinant, biological, or a combination thereof. See, generally, U.S. Pat. No. 6,204,326, issued 20 Mar. 2001, to Cook et al.; U.S. Pat. No. 5,539,083, issued 23 Jul. 1996, to Cook et al.; Tian and Wickstrom (2002) Org. Lett., 4: 4013-4016; Niemeyer (2002) Trends Biotechnol., 20: 395-401; Beier and Hoheisel (1999) Nucleic Acids Res., 27: 1970-1977; Efimov et al. (1999) Nucleic Acids Res., 27: 4416-4426; Koppitz et al. (1998) J. Am. Chem. Soc., 120: 4563-4569; and Misra et al. (1998) Biochemistry, 37: 1917-1925, which are incorporated by reference in their entirety herein. After synthesis or manufacture, the oligonucleotide probes may optionally be purified or isolated. Oligonucleotide probes that are labelled during synthesis may be optionally be checked for signal specificity by preliminary experiments on samples containing the target nucleic acid sequences. If the hybridization signal obtained in these preliminary experiments shows excessive background or non-specific signal, then a replacement oligonucleotide probe may be considered from a different region of the target nucleic acid sequence.

### EXAMPLES

### Example 1

### In situ Hybridization Using Oligonucleotide Probe Sets

This example describes a non-limiting embodiment of a method to detect the relative amounts of at least two target nucleic acid sequences in at least one sample by use of a corresponding set of detectably labelled oligonucleotide probes for each of the at least two target nucleic acid sequences, and detecting the hybridization of each of the corresponding sets of oligonucleotide probes to its respective target nucleic acid sequence. In this specific example, sets of oligonucleotide probes are designed for use to detect and compare the relative amounts of different messenger RNAs (mRNAs) in tissue samples. The two messenger RNAs of interest were IRSp53 and Calsenilin. IRSp53 (a nucleic acid sequence of 1798 nucleotides with the GenBank accession number AF390179, fully described in Thomas et al. (2001) Neurosci. Lett., 309: 145-148, which is incorporated by reference in its entirety herein) is a substrate for the insulin receptor tyrosine kinase. Calsenilin (a nucleic acid sequence of 2711 nucleotides with the GenBank accession number AF 184624, the sequence of which was directly submitted to GenBank on 8 Sep. 1999 by D. G. Jo, M. J. Kim, and Y. K. Jung, Life Science, Kwang-Ju Institute of Science and Technology, Kwang-Ju 500-712, Republic of Korea, and incorporated by reference in its entirety herein) is a calcium-binding protein that interacts with presenilins and is a substrate for caspase-3. The reagents and experimental details are given here as examples and are not intended to suggest limitations of the invention.

Materials and Methods

1. Suggested Time

Tissue preparation: about 2 hours.
Cryostat sectioning depends on the number of transcripts to be labelled and the mapping interval desired, but can take up to about 16 hours including fixation.
Oligonucleotide probe labelling and purification: about 5 hours.
Prehybridization: about 2 hours.
Hybridization: about 18 hours (overnight incubation).
Posthybridization: 4 hours.
Film exposure time from about 1 to about 12 days, depending on the level of mRNA expression.
The entire protocol can run anywhere from about 7 to about 20 days.
   2. Materials
   2.1. Animals

Adult (90 days) male C57 mice (n=3; Charles River) were maintained on a 12-hour light/dark cycle and had free access to food and water.

2.2. Special Equipment

Cryostat: Micron HM560 (Zeiss, Mikron Instruments, San Diego, Calif.); Incubation oven, ThermoIEC Centra-CL2 centrifuge, vortex genie 2, dry bath incubator, Lab Line rotator, film cassettes (Fisher Scientific, Pittsburgh, Pa.); refrigerator (4 degrees Celsius), freezer (−20 degrees Celsius), deep freezer (−80 degrees Celsius) (Fisher Scientific, Pittsburgh, Pa.); Beckman Multi-purpose Scintillation Counter (Beckman Coulter, Fullerton, Calif.); microcentrifuge 5415D (Eppendorf, Westbury, N.Y.); humidity chamber, aluminum tray (ThermoShandon, Pittsburgh, Pa.); slide holder, 250-milliliters staining bucket (VWR, West Chester, Pa.); microscope (Carl Zeiss, Inc., Thornwood, N.Y.); computer-based image acquisition software: NeuroMosaic™ (Neurome, Inc., La Jolla, Calif.); computer-based image analysis system (MCID): Imaging Research (St. Catherines, Ontario, Canada).

2.2. Chemicals and Reagents

Deoxyadenosine 5′ [alpha-³⁵S] thiotriphosphate (dATP) (Amersham-Pharmacia, Piscataway, N.J.); acetic anhydride, Denhardt's-50×, dextran sulfate, dithiothreitol (DTT), ethylenediaminetetraacetic acid (EDTA), formamide, sodium dodecyl sulfate (SDS), sodium chloride, Tris base, xylenes, proteinase K, Kodak biomax MR 18×24 centimeter film (Fisher Scientific, Fair Lawn, N.J.); premixed 20× SSC Buffer, Terminal Transferase, tRNA (Roche, Indianapolis, Ind.); triethanolamine (TEA), Peel-A-Way mold (VWR, Aurora, Ohio); diethylpyrocarbonate (DEPC), DNA from salmon testes, Poly-Prep slides (Sigma, St. Louis, Mo.); Elmer's Rubber Cement (Staples, Columbus, Ohio); CentriSep columns (Princeton Sep, Adephia, N.J.).

2.3. Oligonucleotides

Oligonucleotides may be obtained from any suitable supplier. In this example, the oligonucleotides were purchased from MWG Biotech, Inc. (High Point, N.C.). The oligonucleotides were dissolved in double-distilled H₂O at a concentration of 100 picomoles per microliter and stored at −20 degrees Celsius.

2.4. Solutions

Care was taken to avoid contamination by ribonucleases. The plasticware and glassware was autoclaved and devoted exclusively to in situ hybridization. All solutions for prehybridization and hybridization were prepared using DEPC-treated, double-distilled H₂O (“ddH₂O”) (“DEPC-water”) and were filtered through sterile filters (0.22 micrometers filter system). DEPC-water was prepared by incubating ddH₂O with 0.1% DEPC overnight followed by autoclaving for 30 minutes.

3. Detailed Procedure

3.1. Selection of Oligonucleotides

As provided above in the Detailed Description of the Invention, the present invention provides methods to provide sets of oligonucleotide probes. The following procedure describes a non-limiting example of the method of the invention, as was applied to in situ hybridization on brain tissues.

Multiple, non-overlapping oligonucleotide probes were used to increase sensitivity of the signal (see Trembleau and Bloom (1995) J. Histochem. Cytochem., 43: 829-841, which is incorporated by reference in its entirety herein). For these particular in situ hybridization experiments, between 4-10 oligonucleotide probes could be designed for any given transcript. However, once chosen, the number of probes was preferably kept constant in order for the quantification to be most easily validated.

N, the number of oligonucleotide probes per set, is preferably selected according to the expected abundance or scarcity of the target messenger RNA in the sample. As a general practice, the less abundant the target messenger RNA is, the larger N preferably is. In one embodiment, messenger RNAs can be predicted or expected to be characterized by “low copy number” (about 1 to about 30 copies per cell), “medium copy number” (about 30 to about 1000 copies per cell), or “high copy number” (greater than about 1000 copies to about 100,000 copies per cell). Typical values of N for these ranges are from about 3 to about 4 oligonucleotide probes for “high copy number” messenger RNAs, from about 5 to about 8 probes for “medium copy number” messenger RNAs, and from about 9 to about 12 probes for “low copy number” messenger RNAs. However, these values of N merely serve as guidelines, and the actual N may be varied by the user according to his or her requirements. For example, in one non-limiting embodiment, 6 oligonucleotide probes can be used for comparing medium to high copy number messenger RNAs, and 10 oligonucleotide probes can be used for comparing low copy number messenger RNAs.

Probes for detecting target messenger RNAs can be generated to any published or known cDNA sequence. In choosing a region within the sequence, it is preferable to avoid regions of high homology to other related mRNA sequences, in order to minimize undesirable cross-hybridization of the probe, false signals, and high background. Designing probes using a PCR primer design program may be unnecessary because the oligonucleotide probes are generally longer than PCR primers (and thus not subject to the same stringent hybridization conditions), and because the oligonucleotides are usually chosen from specific non-homologous sequences within the cDNA (and thus amenable to selection by hand).

A general approach to designing sets of oligonucleotide probes according to the method of the present invention is provided in detail above under the heading, “III. METHOD TO PROVIDE SETS OF OLIGONUCLEOTIDE PROBES”. For the purposes of this non-limiting example, the following steps and criteria were found to be generally satisfactory in designing sets of oligonucleotide probes for in situ hybridization:
1. An oligonucleotide length of preferably about 40 bases was chosen.
2. Regions in the sequence that are not GC-rich or AT-rich were selected, with the desired sequence preferably having a GC content between about 45% to about 62%.
3. Runs of more than five identical nucleotides in series were preferably avoided.
4. Because the oligonucleotide probes can be internally labelled during synthesis using a modified amino-allyl-dT, it was preferable to choose a sequence containing 3 adenines at relatively equal spacing intervals, to allow the same oligonucleotide probe sequence to be used for both radioactive and non-radioactive in situ hybridization.
5. The oligonucleotide sequences were selected to permit fewer than about 15 to about 20 nucleotides between adjacent oligonucleotide probes when the adjacent oligonucleotide probes were hybridized to the corresponding target messenger RNA.
6. Each oligonucleotide sequence was be compared against the GenBank database to confirm that it recognized the target mRNA and preferably did not show similar homology to other known mRNAs or genomic sequences.
7. After the oligonucleotide was labelled, the specificity of the signal was checked by preliminary experiments on appropriate microscopy samples. If the hybridization signal from the sections showed excessive background or non-specific signal, a replacement oligonucleotide from a different region of the cDNA was considered.
   3.2. Oligonucleotide Probe Labelling and Purification

Oligonucleotides probes were labelled and purified according to the following procedure.
1. To 1 microliters of a stock oligonucleotide solution (2 picomoles per microliter) was added 2 microliters 5× concentrated reaction buffer for terminal transferase, 1 microliters 25 micromolar cobalt chloride solution, 2 microliters (20 microcuries) deoxyadenosine 5′[alpha-³⁵S] thiotriphosphate, 1 micro liter terminal transferase (25 units), and DEPC-water to a final volume of 10 microliters.
2. The solution was mixed and incubated for 60 minutes at 37 degrees Celsius in a heating block.
3. The reaction was stopped by adding 100 microliters 10 millimolar EDTA, pH 8.0.
4. The oligonucleotide probe was precipitated by adding 2 microliters glycogen, 1 microliter yeast-tRNA (10 milligrams per milliliter), 10 microliters 3 molar sodium acetate, pH 8.0 and 3 volumes (300 microliters) of 100% cold (−20 degrees Celsius) ethanol, mixing, and incubating the mixture at −20 degrees Celsius overnight.
5. The mixture was centrifuged at 14,000 rpm, 4 degrees Celsius for 30 minutes.
6. The supernatant was poured off, and the pellet was rinsed with 200 microliters 70% cold ethanol and centrifuged at 14,000 rpm, 4 degrees Celsius for 10 minutes.
7. The pellet was air-dried for about 30 minutes and resuspended in 100 microliters DEPC-water. The concentration of the labelled oligonucleotide was 20 nanomolar.
8. The oligonucleotide probe was passed over a CentriSep column according to the manufacturer's instructions and the eluate was captured in a clean microfuge tube.
9. The incorporated radioactivity was measured by counting a 1/10 dilution of the oligonucleotide probe in a liquid scintillation counter. An acceptable probe typically yielded about 20,000 to about 100,000 counts per minute per microliter.
10. To ensure stability for several months, oligonucleotide probes were stored at their working concentration in the hybridization solution at −20 degrees Celsius.
   3.3. Hybridization Solution

Hybridization solution for the brain tissues was prepared as follows.
1. A 10-milliliters final volume of hybridization solution was prepared by combining 5 milliliters formamide (pre-aliquoted), 1.1 milliliters 5 molar NaCl, 800 microliters 1 molar Tris, pH 8.0, 80 microliters 0.5 molar EDTA, pH 8.0, 100 microliters 10% SDS, 100 microliters 1 molar dithiothreitol, 200 microliters 50× Denhardt's solution, 20 microliters salmon sperm DNA (10 milligrams per millilter) and 2 milliliters dextran sulfate (50% stock dissolved in DEPC-water, pre-aliquoted; added by displacement).
2. The hybridization solution was completed by adding the entire volumes of all 6 oligonucleotide probes into the mixture. The solution was mixed well and centrifuged at 3000 rpm for 5 minutes at room temperature to remove air bubbles. The final concentration of each oligonucleotide probe was 200 picomolar and total probe concentration was 1.2 nanomolar.
3. To determine non-specific hybridization signal (negative control), 1-2 milliliters of hybridization solution were removed, to which was added each of the unlabelled oligonucleotides (2 picomoles per microliter stock) at 100-fold excess (2 micromolar) of the labelled oligonucleotide probes. The hybridization solutions could be used immediately or stored at −20 degrees Celsius.
   3.4. Tissue Preparation

Brain tissues for use in the in situ hybridization experiments were prepared from the C57 mice as follows.
1. The animals were sacrificed by decapitation.
2. The brain was removed from each animal and frozen in isopentane at −25 degrees Celsius for 30 seconds. The time required to remove the brain from the skull was minimized to prevent mRNA degradation. The tissue was stored at −80 degrees Celsius in airtight plastic bags until use.
3. Before sectioning, the brains were thermally equilibrated on a chuck for 30 minutes in the cryostat (−20 degrees Celsius). At the same time, poly-L-lysine coated slides (Sigma, St. Louis, Mo.) were placed in the cryostat to allow them to cool to the cutting temperature. Brains were cut one at a time, or multiple brains (e.g., three brains) at a time were cut in order to standardize conditions and minimize tissue handling and storage.
4. 20 micrometer thick sections were thaw-mounted onto the slides and kept at −20 degrees Celsius inside the cryostat for at least 15 minutes after the last section was cut to improve tissue adhesion to the slide.
5. Slide-mounted sections were transferred to freshly made 4% paraformaldehyde in 0.1 molar phosphate buffered saline (PBS), pH 7.4 and postfixed at room temperature for 1 hour.
6. The slides were washed 3×5 minutes in PBS and dipped briefly in DEPC-water to remove salts.
7. The fixed, slide-mounted sections were dried in a stream of cold air and stored desiccated at −80 degrees Celsius until use.
   3.5. Prehybridization

The in situ hybridization procedure that is described below was used with the oligonucleotide probes. The procedure used with the riboprobes has been described in detail in Winzer-Serhan et al. (1999) Brain Res. Brain Res. Protoc., 3: 229-241, which is incorporated by reference in its entirety herein. Slides were placed into racks and 250-milliliter staining buckets designated specifically for the prehybridization procedure. All solutions were prepared in DEPC-water and filter-sterilized whenever possible.
1. The slide-mounted sections were thawed to room temperature for at least 30 minutes before removing them from the slide box.
2. The sections were incubated in 0.1 M Tris, pH 8.0, 0.05 M EDTA, pH 8.0 containing 0.1 milligrams per milliliter proteinase K (10 milligrams per milliliter stock, pre-aliquoted) for 10 minutes at room temperature to increase probe penetration into the tissue.
3. The slides were rinsed briefly in DEPC-water and then incubated in 0.1 molar triethanolamine (TEA), pH 8.0 for 2-3 minutes.
4. To block the positive charges on the tissue induced by proteinase K digestion, the slides were incubated in 0.1 molar TEA, pH 8.0 containing 0.25% acetic anhydride for 10 minutes at room temperature with gentle agitation.
5. The sections were washed twice in 2× sodium chloride/sodium citrate (SSC; made from 20× stock) for 2 minutes each at room temperature.
6. The sections were dehydrated through ascending concentrations of ethanol (50, 75, 95 and 2×100%) for 2 minutes each, drained well, and air-dried for 30 minutes in a stream of cold air.
   3.6. Hybridization

As preferred practice, each hybridization experiment included negative control slides, in order to be able to detect non-specific hybridization signals.
1. The hybridization oven was turned on to 42 degrees Celsius before starting the procedure. The aluminum hybridization trays were prepared by taping down wooden dowels to support the slides.
2. The hybridization solution was thawed to room temperature, mixed to dissolve any SDS precipitate that may have formed, and centrifuged at 3000 rpm for 5 minutes to remove air bubbles.
3. Using a sterile pipette tip, 150 microliters of hybridization solution was placed onto a 22×50 millimeter glass cover-slip resting on a dark surface, or 200 microliters was placed onto a 22×60 millimeter cover-slip, depending on the amount of tissue on the slide.
4. A slide was laid slowly face-down on top of the hybridization solution, avoiding the formation of air bubbles, and then both slide and cover-slip quickly turned right-side up.
5. The edges of the cover-slip were sealed with a bead of liquid rubber cement and the slide placed on an aluminum hybridization tray.
6. The tray was placed inside an acrylic humidity chamber, the lid closed, and the sections hybridized for approximately 18 hours in the 42 degrees Celsius oven.
   3.7. Posthybridization

Solutions for the posthybridization procedure were made in ddH₂O. The slides were placed into racks and buckets designated specifically for the posthybridization procedure.
1. The humidity chamber was removed from the oven and the slides cooled to room temperature for 10-20 minutes. Meanwhile, 1 liter of 2× SSC in ddH₂O was prepared for the subsequent washes.
2. Using a sharp pair of forceps, the dried rubber cement was carefully removed from each slide, starting at one of the top corners. The cover-slip generally came off as well, if not, the cover-slip was gently teased off, avoiding any damage to the underlying tissue.
3. The sections were incubated through six solutions of decreasing salinity for 30 minutes each at 42 degrees Celsius with agitation. The sections were washed twice in 2× SSC, once in 1×SSC, once in 0.5× SSC and twice in 0.1× SSC. Then 250 microliters of 1 molar DTT was added to each 250 milliliters dilution of SSC and preheated before using.
4. The sections were dehydrated through ascending concentrations of ethanol (75, 95 and 2×100%) for 2 minutes each, drained well, and air-dried for 30 minutes in a stream of cold air.
5. Slides were now storable at room temperature, protected from dust, or could be taped onto cardboard for film exposure.
   3.8. Preparation of Calibration Standards

Densitometric analysis of in situ hybridization signal can be carried out within an individual experiment or film to provide semi-quantitative comparisons. Relative levels from regional analysis, or inter-conditional variations can be normalized to an internal standard. However, for quantitative densitometry, which allows for more accurate comparisons from one hybridization experiment to another, radiolabelled sections are preferably quantitated, for example, by apposing the sections to film along with a set of calibration standards of known concentration.

To quantitate [³⁵S]-labelled hybridization signal, [¹⁴C] brain paste standards were used, because of the similar radioactive spectral emissions of [³⁵S] and [¹⁴C] and because these standards have a longer shelf life. The standards were prepared from rat brain homogenates as described by Miller (1991) Neurosci. Lett., 121: 211-214, which is incorporated by reference in its entirety herein. Section diskettes were cut at 20 micrometers, which was the standard section thickness used in the hybridization experiments. Eleven brain-paste standards were generated with increasing radioactivity concentrations ranging from 0.02 to 5.38 nanocuries per milligram tissue wet weight (35 to 11,838 disintegrations per minute per milligram). When apposed to Kodak Biomax-MR film, this range provided a signal that was within the linear range of the film from between about 1 to about 8 days of exposure, and was sufficient to calibrate the hybridization signal produced from most mRNA transcripts.

3.9. Autoradiograms

Autoradiography of the hybridized sections was carried out as follows.
1. The slide-mounted sections, along with the [¹⁴C] calibration standards, were apposed to Kodak Biomax-MR film in metal cassettes. The cassettes were stored at 4 degrees Celsius for an appropriate exposure time. The exposure time depended on the expected abundance of the messenger RNAs and the desired image intensity for both qualitative and quantitative analysis. Tissue regions with higher abundance of mRNA, and therefore higher levels of signal were exposed for shorter times than regions with lower levels. Since the dynamic range of film for quantitation is limited to signal that is within 1 unit of optical density (OD), films were generally exposed for several different time periods (from about 1 to about 8 days) and the exposures that best fitted this criterion for the region(s) of interest were chosen. In one example, films were exposed for 2, 4, and 6 days and quantitation was performed on the 6-day film for the oligonucleotide probe signal and on the 2-day film for the riboprobe signal.
2. The films were developed in Kodak D-19 developer (diluted 1:1 with water) for 4 minutes at room temperature under darkroom conditions. The films were washed for 30 seconds in 2% glacial acidic H₂O and fixed for 5 min in Rapid Fix. The films were rinsed in H₂O for 30 minutes and hung to air-dry.
3. At this point, slides could be dipped in emulsion according to the procedure of Winzer-Serhan et al. (1999) Brain Res. Brain Res. Protoc., 3: 229-241, which is incorporated by reference in its entirety herein. This provided for more detailed high-resolution analyses, which were useful in distinguishing the boundaries of labelled regions.
4. Sections were counterstained with cresyl violet, dehydrated through ascending concentrations of ethanol and xylenes, and covered with a cover-slip and DPX mounting medium.
   3.10. Quantification of mRNA Expression

Autoradiographic images and the counterstained sections were digitally acquired using a fully motorized Zeiss Axioplan 2ie microscope equipped with an AxioCam HRc and an 8-slide scanning stage, which was automated by computer-based software, NeuroMosaic™ (Neurome, La Jolla, Calif.) for high-throughput digital image acquisition. Entire sections composed of multiple image tiles were acquired at a final magnification of 66× (5.2 micrometers per pixel). Several software packages, such as MCID (St. Catherines, ON, Canada) are also publicly available that can capture autoradiographic images at lower resolution.

Quantitative analysis of film images was done using a computer-based image analysis system (MCID, St. Catharines, ON, Canada). A calibration curve of relative optical density (ROD) versus radioactivity concentration of the [¹⁴C] standards was constructed by the program and the measurements were best fit to either a third- or fourth-degree polynomial curve. Autoradiographic brain images were digitally aligned with their counterstained Nissl sections. Contours were drawn bilaterally over the basal ganglia (including the caudate putamen, nucleus accumbens, and globus pallidus), the hippocampus (including area CA1/2, CA3, and the dentate gyrus) and the cortex (from the midline to the rhinal fissure) of the Nissl sections. Optical densities were automatically measured from the corresponding regions of the autoradiographic images. Since sections for a given data set were collected at 200-micrometer intervals, the data gathered was from between about 13 to about 25 sections per brain containing these regions. Specific mRNA expression was determined by subtracting the non-specific hybridization signal obtained with the negative controls from the values obtained with the oligonucleotide probes.

3.11. Characterization of Oligonucleotide Probes

Labelled oligonucleotides were characterized by gel electrophoresis to determine their size, the number of incorporated radionucleotides to the 3′ end, and the consistency of labelling. Randomly selected oligonucleotide probes were mixed 1:1 with 2× sample buffer and loaded onto a 10% Tris-borate-EDTA-urea denaturing acrylamide gel along with a 25-base pair DNA stepladder for sizing. Gels were then stained with methylene blue to detect the size markers, dried onto Whatman filter paper, and apposed to Kodak Biomax-MR films for between about 1 hour to about 3 hours.

3.12. Real-Time Quantitative PCR

Quantitation of relative gene expressions was performed by using Real-Time Quantitative PCR using the ABI PRISM 7700 Sequence Detection System (Applied Biosystems, Foster City, Calif.). This method combines PCR, cycle-by-cycle fluorescence detection, and analysis software for high-throughput quantitation of nucleic acid sequences. Reactions are characterized by the cycle number at which amplification of a PCR product is first detected. The higher the copy number of the nucleic acid target, the sooner a significant increase in SYBR I Green fluorescence is observed. Quantitation of the amount of target in the sample is accomplished by measuring the cycle number at which a significant amount of fluorescence product is produced.

For each cDNA template, the cycle threshold (Ct) necessary to detect the amplified product was normalized to the Ct values of a control gene, mouse glyceraldehyde-3-phosphate dehydrogenase (GAPDH), based on the similarity of expression across all sample templates. GAPDH Real-Time PCR primer sequences were selected from a sequence of 1228 nucleotides with the GenBank accession number NM_{—}08084 (Mootha et al. (2003) Cell, 115: 629-640, which is incorporated by reference in its entirety herein). Relative differences in target abundance were estimated by calculating the difference in cycle threshold (delta Ct) at which amplified product was detected. Assuming that each cycle produces a two-fold amplification, the ratio of relative differences in target abundance was calculated using the formula: ratio=2^{(delta Ct)}. The entire process was carried out by the integrated software (SDS v1.7) of the 7700 system.

Reactions were performed in a 30-microliter volume with 5 micromolar Forward and Reverse primers, 50 picograms of cDNA and SYBR® Green PCR Master Mix (Applied Biosystems) with AmpliTaq Gold® DNA Polymerase. Gel analysis by ethidium bromide staining was done to visualize a single band at the correct size to confirm that the SYBR green-bound fluorescence is from the amplicon, and from not primer dimers. Primers for Real-Time Quantitative PCR validation were selected by the integrated software package, Primer Express™, accompanying the ABI PRISM 7700. The templates for Real-Time PCR were cDNAs made from the RNA extracted from the basal ganglia, hippocampus, and cortex of C57BL/6 mice. For each cDNA template, the Ct value necessary to detect the amplified product was normalized to the Ct values of the control gene, mouse GAPDH, based on the similarity of expression across all sample templates. Three separate Real-Time PCR experiments were performed, each containing reactions for both IRSp53 and Calsenilin cDNA templates.

4. Results

4.1. Probe Characterization

Two methods of in situ hybridization, using either oligonucleotide probes or riboprobes, to detect the expression pattern of two separate mRNA transcripts in the brain were compared and the quantitative analysis of the results using an established method of real-time PCR for quantitation of nucleic acid sequences was validated. The two messenger RNAs of interest were IRSp53 and Calsenilin. IRSp53 (a sequence of 1798 nucleotides with the GenBank accession number AF390179, fully described in Thomas et al. (2001) Neurosci. Lett., 309: 145-148, which is incorporated by reference in its entirety herein) is a substrate for the insulin receptor tyrosine kinase. Calsenilin (a sequence of 2711 nucleotides with the GenBank accession number AF184624, sequence directly submitted to GenBank on 8 Sep. 1999 by D. G. Jo, M. J. Kim, and Y. K. Jung, Life Science, Kwang-Ju Institute of Science and Technology, Kwang-Ju 500-712, Republic of Korea, and incorporated by reference in its entirety herein) is a calcium-binding protein that interacts with presenilins and is a substrate for caspase-3.

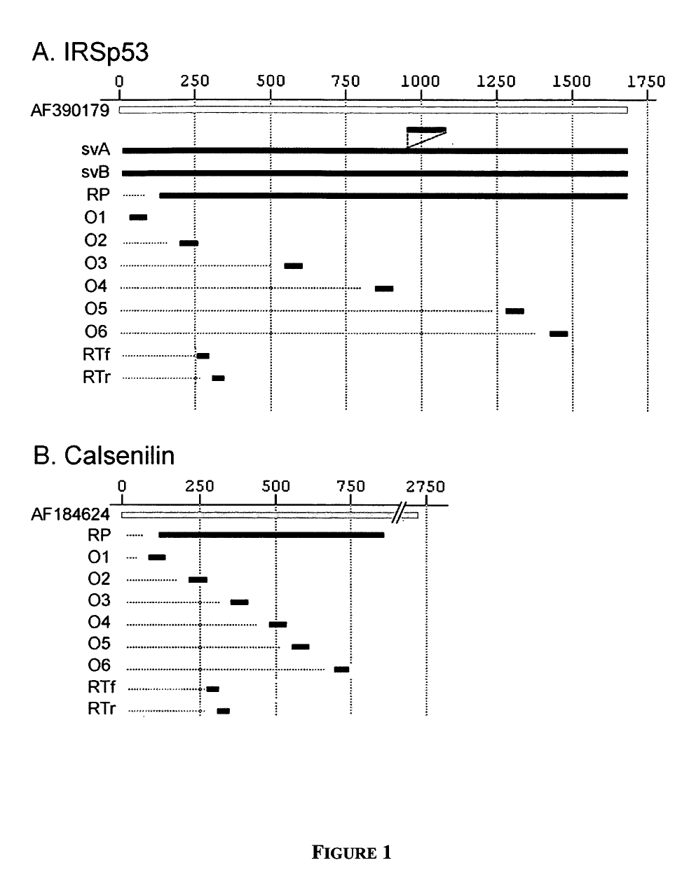

Sets of oligonucleotide probes and riboprobes were both constructed that would overlap in their target sequence as much as possible, while still keeping the constraints necessary for the individual methods. Moreover, both IRSp53 and Calsenilin mRNA transcripts have been shown to have alternative splice variants (Alvarez et al. (2002) J. Biol. Chem., 277: 24728-24734, and Spreafico et al. (2001) Mol. Cell. Neurosci., 17: 1-16, which are incorporated by reference in their entirety herein). The IRSp53 mRNA has two major splice variations within exon 9 named “A” and “B” (FIG. 1), while the Calsenilin mRNA has two variations within exon 3. An unbiased sampling of all splice variants for a given mRNA was provided.

For the target IRSp53 messenger RNA, one IRSp53 riboprobe (SEQ ID No. 1) and six IRSp53 oligonucleotide probes numbered 1 through 6 (SEQ ID No. 2 through SEQ ID No. 7) were prepared. IRSp53 forward (SEQ ID No. 8) and reverse (SEQ ID No. 9) Real-Time PCR primers were also prepared. For the target Calsenilin messenger RNA, one Calsenilin riboprobe (SEQ ID No. 10) and six Calsenilin oligonucleotide probes numbered 1 through 6 (SEQ ID No. 11 through SEQ ID No. 16) were prepared. Calsenilin forward (SEQ ID No. 17) and reverse (SEQ ID No. 18) Real-Time PCR primers were also prepared. In addition, forward (SEQ ID No. 19) and reverse (SEQ ID No. 20) Real-Time PCR primers were prepared for the control gene, GAPDH. The relative positions of the riboprobes, oligonucleotide probes, and Real-Time PCR primer sets utilized for IRSp53 and Calsenilin are illustrated in FIG. 1. Table 1 gives the sequences for the riboprobes, oligonucleotide probes, and Real-Time PCR primers used for IRSp53 and Calsenilin, as well as the sequences for the GAPDH Real-Time PCR primers. All sequences in Table 1 are given in the 5′ to 3′ direction.

TABLE 1

| | | Nucleotide | SEQ |
|---|---|---|---|
| IRSp53 Probes | | position in | ID |
| and Primers | Sequence | IRSp53 | No. |
| riboprobe | AGGAACGGCCACGCUGUAGGGUCUCUGCUUGAAUGUGCCGGCU | 138-1552 | 1 |
| | GUCUGGGUGGGGAAUGCCCGGGAGGACGUGCCGUAGUCAGGAG | | |
| | GGGGGAGGGCCAGGUCAUCCUUGUCUAGGAGGUUGCCUGUGCU | | |
| | GCUGCUCUUGCCCUGCUGCAGGCUCAUAUGCAAUCUGUCACUU | | |
| | CCGUCACUGUCCAGGACCCGGGUGUAGGAGAAGGGGAACCAGC | | |
| | CCCGCAUCUUGGUCUUCUCACUCUCCCCAUAGUGCCAGCCGUC | | |
| | ACGGGCCUCAGGCACUAGCAGCGUGAUGAGGUCGCCCUCCUUG | | |
| | AAGCUCAGCAGAGUGCUAUUGUCAGCGGCCGCGUGGGAGAAAA | | |
| | UGGCCUUGACCCGCAUGCGGCCGUUACGUUCUAGGCCAGCUGC | | |
| | CAUGGAGCUUGAACGUGGCAGUGUCUUGUUCUCAGUGGUGGCA | | |
| | UAGCUGUUCUUCGGCGUCACGCUCUUGCGCACGGGGAGUGUGU | | |
| | UGGAGUACGAGUCGCUCAGCUUGCUUUGAGACUGCGGGGGAGA | | |
| | CAGGGAUUUGGGCUGGGCAGCCUUUCGGUCAGCCCAGGGGUUG | | |
| | UAGUCCUCGCUGUCCGGGCCUGCGACGCCAUUCAUGACAGGUA | | |
| | CAUUCUCCUGAGCAGACAUCCGCCCCACAAAUGGUGCCAGCUC | | |
| | AGGUGGCACUGGCAAGGGCUUGGCUCCAGGAAUGGGAUCUGAG | | |
| | AUGACCAGGUUGGACUUGGAAGCUGACAGGGCACUUGGAAGGA | | |
| | UGGAGCCAUUGCUAUUGGCCAUCUGUUGCAUCAGCUGGACAGC | | |
| | ACGGUCUGGGAUCUUGUUGGGGUCGGCACAGGCUUGCUGCCAC | | |
| | AGAGGCAGCUUCUGGGCCAGCAACUCCUUGCCCUUGGAAUGGU | | |
| | AAGCAGCAGAGUUCUUGGCCACAGCGCACUGCUUUUCCACCAG | | |
| | GAAGCAGAACCUCCUGCGCUCCUCAGUGAGUGCUGUCUUGUAG | | |
| | CCGUCAGACACGUAGUUCUCCAGCUCGCCCUGCUUAUUGCUGA | | |
| | UGGCAUCGAUGUACUGCAGCUCCUUGUCUGAGUACUUCUGAGG | | |
| | GUUCUUACUCCCUUGGCUCUUCUUGCGGAGCUUCUUCAGCUCA | | |
| | GCCUGACACUUGUCCAGGGCGUCCCCUUUGCUCCUCUGUUCCG | | |
| | UUUGGUAUUUCUUCAGUGCAGCACUUAGAUACCUGGAGUCCAG | | |
| | UUCUACUUUCUGCUCCAGCUGCGUGAGCAGCUCAUUGUGAAAA | | |
| | GACUUCAGCGUCUCUUCCAACUGGUUCUGGAUCUGCCGGUGCA | | |
| | CCUCAGCCAUCUGGAAGAGGACGUCCCCAAGUUCCUUAGAGCC | | |
| | CUGGCUCUCGCUGGCCAGCUCCCCCAUCUUUACCAGAGCAUCG | | |
| | AAAUAGCCUUUGGCAGCGAAGGUGACACCUGCCAGUGCUUUCU | | |
| | CAUAGUUCUUGCCCAUGGCGAUGAAGUUGCGGAGGCUGG | | |
| oligonucleotide | TCCGAGCGTGAAAGCGACATGGTCCTGGGTCCCGGCTACA | 42-81 | 2 |
| probe 1 | | | |
| oligonucleotide | CCCATCTTTACCAGAGCATCGAAATAGCCTTTGGCAGCGA | 201-240 | 3 |
| probe 2 | | | |
| oligonucleotide | TCGCCCTGCTTATTGCTGATGGCATCGATGTACTGCAGCT | 543-582 | 4 |
| probe 3 | | | |
| oligonucleotide | GAATGGGATCTGAGATGACCAGGTTGGACTTGGAAGCTGA | 839-877 | 5 |
| probe 4 | | | |
| oligonucleotide | TAGCAGCGTGATGAGGTCGCCCTCCTTGAAGCTCAGCAGA | 1240-1279 | 6 |
| probe 5 | | | |
| oligonucleotide | GCTCTTGCCCTGCTGCAGGCTCATATGCAATCTGTCACTT | 1381-1420 | 7 |
| probe 6 | | | |
| Real-Time PCR | GCCAGGGCTCTAAGGAACTTG | 258-278 | 8 |
| forward primer | | | |
| Real-Time PCR | GCACCTCAGCCATCTGGAAG | 289-308 | 9 |
| reverse primer | | | |
| Calsenilin | | Nucleotide | SEQ |
| Probes and | | position in | ID |
| Primers | Sequence | Calsenilin | No. |
| riboprobe | GACGUUCUCAAACAGCUGCAUGGAGUUCGUGAUGUUCUCAUCC | 111-853 | 10 |
| | UUCUGACAAGUCUCCAGAAAUUCAUCAAUGGUCACCACUCCAU | | |
| | CCUGGUUCCUGUCCAUUUUCUGAAAGAACCUCUCCACAUGCUC | | |
| | CAGGGGUGCAUCCUCCCGCAGGAUGGGGUAGGUGUGGCGGCCG | | |
| | AUCAUGUCGUAGAUGGACUUCAUGAUGGCCAGCAUCUCCUCCU | | |
| | UGGUGAUGCAACCAUCCUUGUUAAUGUCAUAGAGAUUGAAGGC | | |
| | CCACUUGAGCUUCUCAUGGACCGUCCCUCGAAGCAGGAUGGAG | | |
| | AGCCCAACCACAAAGUCCUCAAAGUGGAUGGCCCCGUUCCCAU | | |
| | CAGCAUCGAAGGCAUUGAAGAGGAAGUGUGCAUAGGUGGUGG | | |
| | CAUCUCCCUGAGGGAAGAACUGGGAAUAAAUGAGUUUGAAGG | | |
| | UGUCUUCAUCCACCAGGCCUGUGGGACACUCAUUCUUGAAGCC | | |
| | UCGGUAAAGGGACUGCAGCUCCUUCUUGGUGAACUUGGUCUGA | | |
| | GCUUGUAGCUGGUCCAAGCCCUCUGGCUGAUGGCGCACCGUGG | | |
| | AUAACUCUAGUUCACUGUCACUGCUGUCUGAGCCUUGUGGGGC | | |
| | AGCACUGGACAGGAUCCACUUGAUUAAGCAGCAACGCAUCAGG | | |
| | GCCUGGCGGGUGAACCGUGGCCUUUGCCACUUGAUGCUUUCCC | | |
| | UCUUGCUCAGUGGUAUGCGCCCAGGAUCUCCCAGGAGGUUGCC | | |
| | AUCUGAUGCCUUCA | | |
| oligonucleotide | TGCCATCTGATGCCTTCACGGCTTCCTTGGTCCTCTGCAT | 89-128 | 11 |
| probe 1 | | | |
| oligonucleotide | TGGGGCAGCACTGGACAGGATCCACTTGATTAAGCAGCAA | 220-259 | 12 |
| probe 2 | | | |
| oligonucleotide | ATTCTTGAAGCCTCGGTAAAGGGACTGCAGCTCCTTCTTG | 355-394 | 13 |
| probe 3 | | | |
| oligonucleotide | CCCGTTCCCATCAGCATCGAAGGCATTGAAGAGGAAGTGT | 481-520 | 14 |
| probe 4 | | | |
| oligonucleotide | CCCACTTGAGCTTCTCATGGACCGTCCCTCGAAGCAGGAT | 557-596 | 15 |
| probe 5 | | | |
| oligonucleotide | GAAAGAACCTCTCCACATGCTCCAGGGGTGCATCCTCCCG | 707-746 | 16 |
| probe 6 | | | |
| Real-Time PCR | CTAGAGTTATCCACGGTGCGC | 287-308 | 17 |
| forward primer | | | |
| Real-Time PCR | TTGTAGCTGGTCCAAGCCCT | 318-338 | 18 |
| reverse primer | | | |
| | | Nucleotide | SEQ |
| GAPDH | | position in | ID |
| Primers | Sequence | GAPDH | No. |
| GAPDH Real- | CCCTCACAATTTCCATCCCA | 1130-1149 | 19 |
| Time PCR | | | |
| forward primer | | | |
| GAPDH Real- | TCCCTAGGCCCCTCCTGTTA | 1161-1180 | 20 |
| Time PCR | | | |
| reverse primer | | | |

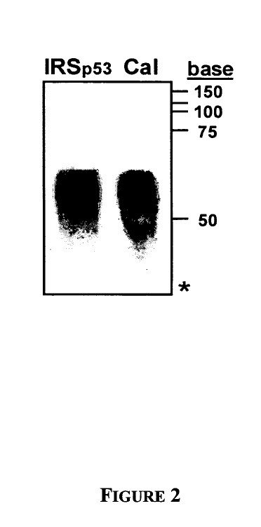

Labelled oligonucleotide probes were also characterized by gel electrophoresis to determine their size, the number of incorporated radionucleotides to the 3′ end, and the consistency of labelling (FIG. 2). A single band of approximately 55-58 bases in size was evident on lanes loaded with either IRSp53 or Calsenilin oligonucleotide probes, suggesting the addition of about 15-18 [³⁵S]-labelled nucleotides. The consistency of labelling for each oligonucleotide probe was similar, with a single discrete band and no major fragmentation. Although some differences in band intensity were observed (FIG. 2), these differences had little or no correlation with the level of incorporated radioactivity determined by liquid scintillation counting.

4.2. Expression of IRSp53 and Calsenilin mRNA

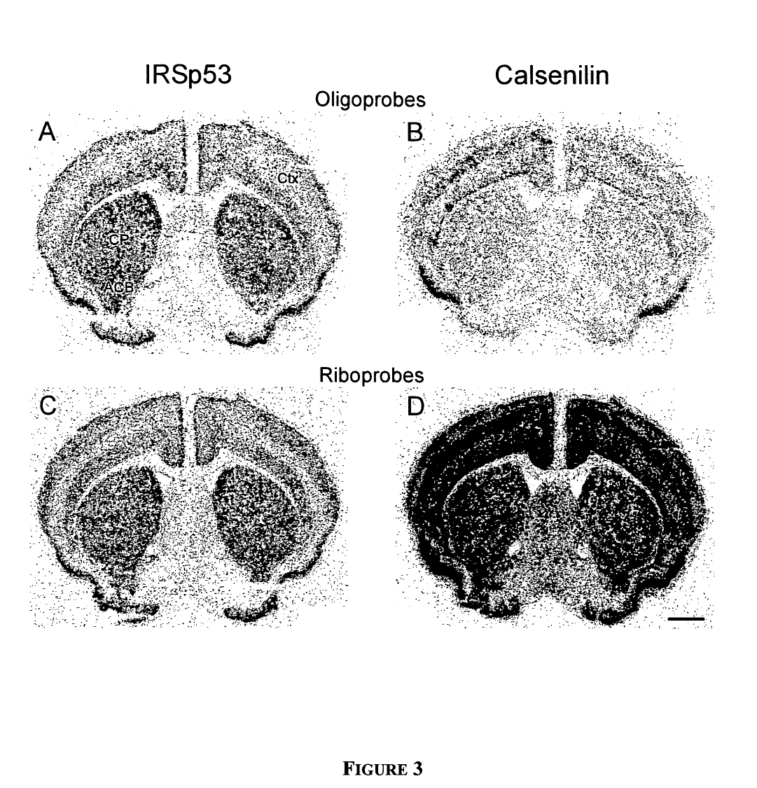

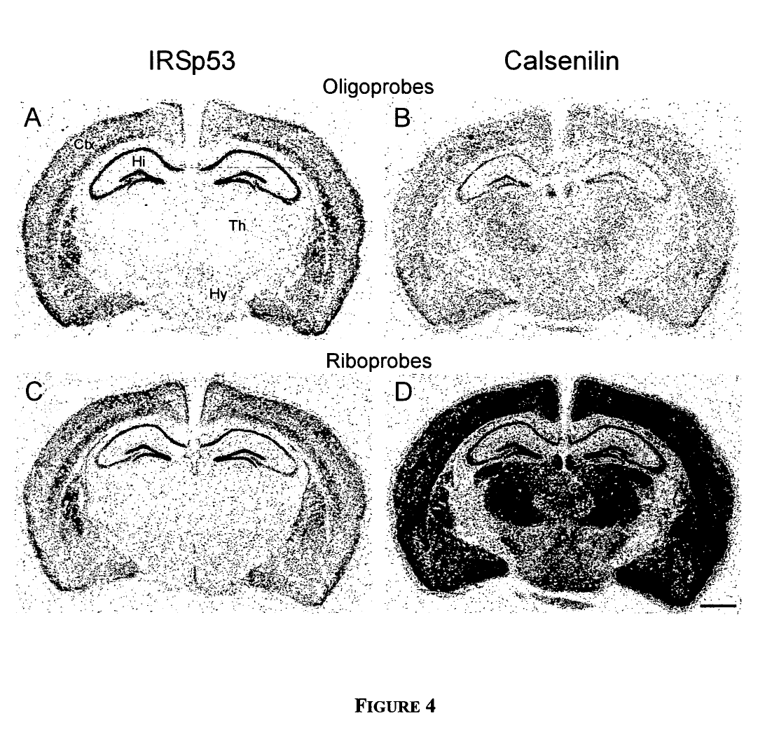

To compare the present oligonucleotide probe-based ISH procedure and an established riboprobe-based procedure (Winzer-Serhan et al. (1999) Brain Res. Brain Res. Protoc., 3: 229-241, which is incorporated by reference in its entirety herein), the expression patterns of two relatively medium abundance mRNA transcripts with known distribution patterns were analyzed. IRSp53 is a substrate for the insulin receptor tyrosine kinase and is involved in cytoskeletal dynamics and mechanisms of neurodegeneration (Okamura-Oho et al. (2001) Biochem. Biophys. Res. Commun., 289: 957-960, which is incorporated by reference in its entirety herein). Autoradiographic analysis revealed high levels of IRSp53 mRNA expression in the olfactory bulb, olfactory tubercle, basal ganglia, and hippocampus, with more moderate levels in the cerebellum and throughout the cortex (FIGS. 3 and 4). Less abundant expression was detectable in such regions as the septum, hypothalamus, and the amygdala, while little or no mRNA expression was detected in thalamic nuclei, the colliculi, and the medulla. In the hippocampus, the hybridization signal was localized to the pyramidal layers of CA1 to CA3, and in the granular layer of the dentate gyrus. Two distinct bands with higher expression levels were also visible in the cortex, corresponding to layers VI and IV-II. The patterns of IRSp53 mRNA expression obtained from the oligonucleotide probe-based and riboprobe-based ISH procedures were identical throughout the brain and similar to previously published results (Thomas et al. (2001) Neurosci. Lett., 309: 145-148, which is incorporated by reference in its entirety herein). Adjacent sections used for the negative control showed hybridization levels that were similar to film background.

Calsenilin is a calcium-binding protein that interacts with presenilins and is a substrate for caspase-3 (Choi et al. (2001) J. Biol. Chem., 276: 19197-19204, which is incorporated by reference in its entirety herein). Qualitative analysis of Calsenilin mRNA expression revealed relatively high levels in the anterior olfactory nucleus, piriform cortex, hippocampus, thalamus, cerebellum, and throughout the cortex, with more moderate levels of expression in the olfactory bulb and basal ganglia (FIGS. 3 and 4). Relatively lower levels of mRNA expression were observed in the septum, hypothalamus, the colliculi, and the medulla. As with the IRSp53 mRNA, expression of Calsenilin mRNA in the hippocampus was localized to the pyramidal layers of CA1 to CA3, and in the granular layer of the dentate gyrus. However, in the cortex, a more distinct band of Calsenilin mRNA expression was observed in layers V, complementary to the pattern of IRSp53 mRNA expression in this region. The patterns of Calsenilin mRNA expression obtained from the oligonucleotide probe-based and riboprobe-based ISH procedures were identical throughout the brain and corresponded to the expression of Calsenilin detailed elsewhere (Hammond et al. (2003) Brain Res. Mol. Brain Res., 111: 104-110, and Spreafico et al. (2001) Mol. Cell. Neurosci, 17: 1-16, which are incorporated by reference in their entirety herein). Adjacent sections used for the negative control showed hybridization levels that were similar to film background.

Quantitative analysis was performed on three major regions in the brain: the basal ganglia, cortex, and hippocampus (FIGS. 3 and 4). Table 2 gives messenger RNA density data (expressed in nanocuries per milligram wet tissue weight) obtained from the two in situ hybridization methods. The data were gathered bilaterally from 13-25 sections per brain and represent the mean±S.E.M. for three animals.

TABLE 2

| | In situ hybridization messenger | | | |
|---|---|---|---|---|
| | RNA density (nanocuries per | | | |
| | milligram wet tissue weight) | | | |
| | OLIGONUCLEOTIDE | | | |
| Brain | PROBE SETS | | RIBOPROBES | |
| region | IRSp53 | Calsenilin | IRSp53 | Calsenilin |
| Basal | 0.175 ± 0.020 | 0.051 ± 0.006 | 0.296 ± 0.021 | 0.529 ± |
| ganglia | | | | 0.059 |
| Hippo- | 0.085 ± 0.015 | 0.031 ± 0.000 | 0.122 ± 0.025 | 0.557 ± |
| campus | | | | 0.016 |
| Cortex | 0.103 ± 0.017 | 0.074 ± 0.004 | 0.175 ± 0.014 | 0.995 ± |
| | | | | 0.060 |

In all three regions, levels of IRSp53 mRNA expression were approximately 1.5-fold higher with the riboprobe-based ISH procedure than with the oligonucleotide probe-based procedure (Table 2), despite the fact that the riboprobe covered a longer (5.4-fold) section of the mRNA transcript than the 6 oligonucleotide probes. Moreover, hybridization with shorter pieces of the IRSp53 riboprobe, obtained by subjecting the probe to alkaline hydrolysis (Cox et al. (1984) Dev. Biol., 101: 485-502, which is incorporated by reference in its entirety herein) in order to increase probe penetration to the tissue, did not increase the signal intensity. However, the trend in regional expression levels was similar between the two methods, with the basal ganglia showing the highest levels of IRSp53 mRNA expression, followed by the cortex, and finally the hippocampus. In contrast, levels of Calsenilin mRNA expression were 10- to 17-fold higher with the riboprobe-based ISH procedure than with the oligonucleotide probe-based procedure (Table 2), despite the fact that the riboprobe only covered a 3.1-fold longer section of that mRNA transcript than the 6 oligonucleotide probes. Furthermore, the trend in regional expression levels between the two methods was slightly different. For the oligonucleotide probe-based method, the cortex displayed the highest levels of Calsenilin mRNA expression, followed by the basal ganglia, and finally the hippocampus; for the riboprobe-based method, the cortex was highest, followed by the hippocampus, and finally the basal ganglia. Together, these results showed a clear difference between the riboprobe-based and the oligonucleotide probe-based ISH procedures that could not simply be explained by the different probe lengths or the length of film exposure. This difference was specifically noted for the Calsenilin mRNA expression levels, which demonstrated a large discrepancy between the two methods.

4.3. Comparison to Real-Time Quantitative PCR

In order to determine which set of data was valid, and thus establish the relative abundance of IRSp53 versus Calsenilin mRNA in the brain, the ISH results obtained by the present method were compared to those obtained by Real-Time quantitative PCR. The PCR method for quantitation of nucleic acid sequences has been used previously to determine the relative abundance of mRNAs in the nervous system (Hu et al. (2002) J. Biol. Chem., 277: 44462-44474, and Schmid et al. (2002) J. Neurochem., 83: 1309-1320, which are incorporated by reference in their entirety herein). Because of the nature of the Real-Time PCR data, the results were not averaged and are presented for each of three individual experiments. Additionally, two separate sets of primers were utilized for the Calsenilin template, which gave similar relative results. However, only one of those primer sets was used for the final analysis, since those data showed less variability between the three PCR experiments.

Table 3 gives the relative levels of IRSp53 and Calsenilin messenger RNAs obtained from three different methodologies: in situ hybridization using sets of oligonucleotide probes, in situ hybridization using riboprobes, and Real-Time PCR. The in situ hybridization ratios were calculated from mean values (Table 2), which were averaged from three individual animals. The Real-Time PCR data are expressed as delta/delta ratios of IRSp53 mRNA to Calsenilin mRNA and were obtained from three individual experiments. In general, the results from the Real-Time PCR experiments demonstrated that IRSp53 mRNA is more abundant than Calsenilin mRNA in all three brain regions examined (Table 3). In the basal ganglia, the levels of IRSp53 mRNA were 2.4 to 2.7-fold higher than Calsenilin mRNA, while in the hippocampus, levels were 1.5 to 2.0-fold higher. Finally, in the cortex, the levels of IRSp53 mRNA were 0.7 to 1.2-fold higher than Calsenilin mRNA.

TABLE 3

Ratio of Insulin Receptor
Substrate p53 messenger
RNA to Calsenilin
messenger RNA

In situ
Hybridization

| Brain | Oligonucleotide | | Real-Time |
|---|---|---|---|
| region | probes | Riboprobe | PCR |
| Basal ganglia | 3.41 | 0.56 | 2.73 |
| | | | 2.60 |
| | | | 2.45 |
| Hippocampus | 2.77 | 0.18 | 2.05 |
| | | | 1.55 |
| | | | 1.71 |
| Cortex | 1.39 | 0.22 | 1.20 |
| | | | 0.73 |
| | | | 0.76 |

When compared to the Real-Time PCR results, the oligonucleotide probe-based ISH method showed almost identical relative levels of IRSp53 to Calsenilin mRNA in all three of the brain regions analyzed (Table 3). The basal ganglia showed 3.4-fold higher levels of IRSp53 versus Calsenilin mRNA, while in the hippocampus levels were 2.8-fold higher. Finally, the cortex exhibited 1.4-fold higher levels of IRSp53 versus Calsenilin mRNA. Additionally, the oligonucleotide probe method demonstrated a similar trend in the ratios of relative abundance as the Real-Time PCR, with the basal ganglia showing the highest ratio, followed by the hippocampus, and finally the cortex. In contrast, the riboprobe-based ISH method showed lower relative levels of IRSp53 compared to Calsenilin mRNA in all three regions, ranging from 0.56-fold difference in the basal ganglia to 0.18-fold difference in the hippocampus (Table 3). Furthermore, the regional trend in ratios for the riboprobe method was different than for the Real-Time PCR. In general, it was apparent from both the SQuISH™ and Real-Time PCR results that Calsenilin mRNA is less abundant than IRSp53 mRNA in various regions of the brain. These data suggest that the ISH procedure using corresponding sets of detectably labelled oligonucleotide probes for each of the target messenger RNAs provides a more accurate representation of relative levels of the target messenger RNAs in the brain microscopy samples, given its concordance with the Real-Time PCR results, which also demonstrated that there are higher levels of IRSp53 mRNA transcripts in a number of brain regions compared to Calsenilin transcripts.

5. Discussion

The present invention presents an in situ hybridization method for standardized quantitative analysis of mRNA transcripts within discrete regions of the brain. The method utilizes a set number of defined oligonucleotide probes for each messenger RNA of interest, allowing for quantitative comparisons, not only for a single mRNA, but also between multiple mRNAs. This has not been possible with previous ISH methods, mainly because of the variations between the different probes used. The SQuISH™ method was compared to an established riboprobe-based ISH method (Winzer-Serhan et al. (1999) Brain Res. Brain Res. Protoc., 3: 229-241, which is incorporated by reference in its entirety herein), and the comparative results were validated by quantitative Real-Time PCR. The PCR technique used in the present invention has been a reliable measure for analyzing relative mRNA levels (Hu et al. (2002) J. Biol. Chem., 277: 44462-44474, and Schmid et al. (2002) J. Neurochem., 83: 1309-1320, which are incorporated by reference in their entirety herein) and the obtained results confirmed the accuracy of the SQuISH™ method.

There are several advantages to using corresponding sets of oligonucleotide probes as opposed to riboprobes for ISH. (1) These probes can be generally designed by any laboratory with access to sequence databases such as GenBank. Familiarity and access to molecular biological techniques, which are usually necessary for the design and production of riboprobes, is not required. (2) ISH procedures using oligonucleotide probes are usually less involved. (3) Oligonucleotide probes can be designed to any suitable length as described above under the heading, “III. METHOD TO PROVIDE SETS OF OLIGONUCLEOTIDE PROBES”. The relative shorter length of oligonucleotide probes (compared to riboprobes) renders issues of tissue penetration by the probes minor and provides for more standardized probe and hybridization conditions. (4) Unlike riboprobes, oligonucleotide probes can be directed to any region of the target sequence, which makes it much easier to avoid areas of homology and unwanted non-specific hybridization. Moreover, the probes can be targeted to identify and quantitatively compare mRNA splice variants, such as those observed for IRSp53 (Alvarez et al. (2002) J. Biol. Chem., 277: 24728-24734, which is incorporated by reference in its entirety herein).

In contrast, riboprobe length is difficult to standardize without extensive and impractical molecular manipulations of the starting DNA template. For the same reason, many riboprobes are relatively long and often encompass regions of homology to other mRNA sequences. This can often lead to cross-hybridization and ultimately higher background signal (Winzer-Serhan et al. (1999) Brain Res. Brain Res. Protoc., 3: 229-241, which is incorporated by reference in its entirety herein). Although in this instance the IRSp53 riboprobe did not appear to have difficulties in penetrating the tissue (since alkaline hydrolysis of the approximately 1.4 kilobase probe did not substantially change the signal intensity), in general, the greater length of riboprobes can affect tissue penetration. Furthermore, the inevitable length of some riboprobes may result in hybridization difficulties due to unknown secondary structure of the target mRNA. This and other issues may have led to the results observed with the long riboprobe used for Calsenilin in this example.

5.1. Optimization

The present invention provides a general approach to designing sets of oligonucleotide probes as provided in detail above under the heading, “III. METHOD TO PROVIDE SETS OF OLIGONUCLEOTIDE PROBES”. In this non-limiting example, six oligonucleotide probes for a given mRNA were tested, together and individually, to examine the specificity of the signal. Although each oligonucleotide was checked against the GenBank database, there were instances when one or more of the six probes demonstrated increased background as manifested by non-specific signal in the white matter or in neuron-rich structures. In those situations, a replacement oligonucleotide could be synthesized.

Once labelled, radioactive oligonucleotide probes should be stored at their working concentration in the hybridization solution. Increased background signal was found when concentrated stock oligonucleotide probes were used after 2-3 weeks of storage. In contrast, oligonucleotide probes kept in working solution were observed to be usable for up to several months after synthesis.

Due to the relative stringency of many ISH procedures, it is generally preferable to use coated slides (for example, slides coated with poly-L-lysine, available commercially from Sigma, St. Louis, Mo.) for good tissue adhesion.

Tissue, both whole and sectioned, is preferably stored at −80 degrees Celsius in order to enhance preservation of the mRNA, especially when working with fresh-frozen tissue. Storage at higher temperatures, even of well-perfused tissue, can result in decreased hybridization signal.

5.2. Additional Protocols

Studies using oligonucleotide probe-based ISH procedures have relied on the use of single oligonucleotide probes to detect mRNA expression. See, for example, Key et al. (2001) Brain Res. Brain Res. Protoc., 8: 8-15, and C. Le Moine, “Quantitative in situ hybridization using radioactive probes to study gene expression in heterocellular systems”, in I. A. Darby (editor), “In situ hybridization protocols”, Humana Press, Totowa, N.J., 2000, pp. 143-156, which are incorporated by reference in their entirety herein. Such mRNA detection may be straightforward when analyzing high abundance mRNAs, but can become impractical when the mRNA of interest is lower in abundance (for example, in the case of many transcripts encoding receptors). Investigators have generally relied on riboprobes for detecting lower-abundance mRNAs, as riboprobes can have a higher specific activity due to their longer length and can offer greater sensitivity. However, the present SQuISH™ method, a non-limiting example of the method of the present invention, uses sets of oligonucleotide probes and provides methods of detecting and comparing the expression of lower-abundance messenger RNAs by simply increasing N, the number of oligonucleotide probes in a corresponding set of oligonucleotide probes directed to a particular target messenger RNA, and thereby increasing the sensitivity. In a non-limiting example, N included between about 8 to about 10 separate oligonucleotide probes for detection of relatively low-abundance receptor messenger RNAs, and provided the ability to detect and quantitatively compare the relative abundance of these scarce transcripts.

The present SQuISH™ method, a non-limiting example of the method of the present invention, can also be used for non-radioactive analysis of mRNA expression. For example, oligonucleotide probes can be labelled by digoxin- (DIG-) or biotin-dATP according to the manufacturer's instructions. Tissue sections can be taken through the same method, followed by immunochemical or histochemical detection. DIG- or biotin-labelled oligonucleotide probes preferably should not be column purified. The oligonucleotides can also be internally labelled, for example, with modified amino-allyl-dT's during synthesis. This can allow variations in the type of label used, and can be advantageous for multiple labelling procedures (depending on the number of labelling sites per oligonucleotide probe), for example, double-labelling with fluorescent tags. Non-radioactive ISH staining provides flexibility for detailed cellular analysis of mRNA expression and can be used in conjunction with the radioactive SQuISH™ for an even more comprehensive study.

The SQuISH™ method, a non-limiting example of the method of the present invention, is particularly suited for use on fresh-frozen tissue. It can permit simultaneous studies for examining both mRNA and protein for most gene products. For example, slide-mounted tissue sections kept inside the cryostat during cutting can be post-fixed and used for ISH, enzyme histochemistry, and immunohistochemistry, while fresh sections sitting outside the cryostat can subsequently be used for receptor binding. With concurrent immunohistochemistry, fixed tissue is generally preferred over fresh-frozen tissue, particularly when morphological preservation is an issue (Cloez-Tayarani and Fillion (1997) Brain Res. Brain Res. Protoc., 1: 195-202, which is incorporated by reference in its entirety herein). However, combined methods have successfully used fresh-frozen tissue (see, for example, C. Le Moine, “Quantitative in situ hybridization using radioactive probes to study gene expression in heterocellular systems”, in I. A. Darby (editor), “In situ hybridization protocols”, Humana Press, Totowa, N.J., 2000, pp. 143-156, and Newton et al. (2002) Brain Res. Brain Res. Protoc., 9: 214-219, which are incorporated by reference in their entirety herein).

### EXAMPLE 2

### A Quick Procedure For In situ Hybridization Using Oligonucleotide Probe Sets

This example describes a non-limiting embodiment of a method to detect the relative amounts of at least two target messenger RNAs in at least one microscopy sample by use of a corresponding set of detectably labelled oligonucleotide probes for each of the at least two target messenger RNAs, and detecting the hybridization of each of the corresponding sets of oligonucleotide probes to its respective target messenger RNA. In this example, the at least one microscopy samples include brain tissues. The reagents and experimental details are given here are as examples and are not intended to suggest limitations of the invention.

Oligonucleotide Probe Selection

Select 6 oligonucleotide probes for any given medium abundance mRNA according to the described constraints. Check each oligonucleotide probe sequence against the GenBank database to confirm that it recognizes the target mRNA. There are many companies providing oligonucleotide synthesizing services.

Oligonucleotide Probe Labelling and Purification

Label the oligonucleotide probes with [alpha-³⁵S]-dATP, precipitate with ethanol, and purify by passing the oligonucleotide probes through a column. Store the labelled oligonucleotide probes at their working concentration in hybridization solution at −20 degrees Celsius.

Tissue Preparation

Sacrifice animals (preferably by decapitation). Quickly remove the brains and freeze them in isopentane at −25 degrees Celsius for 30 seconds. Section the brains (20 micrometers) in a cryostat and thaw-mount onto poly-L-lysine coated slides kept at −20 degrees Celsius. Fix tissue sections with 4% paraformaldehyde, wash and store desiccated at −80 degrees Celsius until use.

Prehybridization

Thaw the slide-mounted sections and treat with proteinase K (0.1 milligram per milliliter) for 10 minutes at room temperature to increase probe penetration into the tissue. Acetylate the tissue for 10 minutes at room temperature with agitation, wash, and dehydrate.

Hybridization

Place 150 microliters of hybridization solution containing 200 picomoles per liter of each oligonucleotide probe on a 22×50 millimeter cover-slip. Slowly lay a slide face down on top of the solution and quickly turn both slide and cover-slip right-side-up. Seal the edges of the cover-slip with a bead of liquid rubber cement and place the slide on an aluminum hybridization tray. Place the tray inside an acrylic humidity chamber and hybridize the tissue sections for 18 hours in a 42 degrees Celsius oven.

Posthybridization

Using a pair of sharp forceps, carefully peel off the dried rubber cement and the cover-slip from each slide. Wash the sections twice in 2× SSC, once in 1× SSC, once in 0.5× SSC and twice in 0.1× SSC for 30 minutes each at 42 degrees Celsius with agitation. Dehydrate through ascending ethanol concentrations and dry the sections for 30 minutes in a stream of cold air.

Signal Detection

Appose the slide-mounted sections, along with [¹⁴C] calibration standards to Kodak Biomax-MR film and store in metal cassettes at 4 degrees Celsius for an appropriate length of time. Develop the films under appropriate conditions. Sections can either be dipped in emulsion for more detailed high-resolution analysis, or immediately counter-stained with cresyl violet.

All publications, including patent documents and scientific articles, referred to in this application and the bibliography and attachments are incorporated by reference in their entirety for all purposes to the same extent as if each individual publication were individually incorporated by reference.

All headings are for the convenience of the reader and should not be used to limit the meaning of the text that follows the heading, unless so specified. Various changes and departures may be made to the present invention without departing from the spirit and scope thereof. Accordingly, it is not intended that the invention be limited to that specifically described in the specification or as illustrated in the drawings, but only as set forth in the claims.

1. A method to detect the relative amounts of at least two target nucleic acid sequences in at least one sample, comprising
(a) providing at least one sample suspected of containing said at least two target nucleic acid sequences;
(b) providing a corresponding set of oligonucleotide probes for each of said at least two target nucleic acid sequences, wherein each of said corresponding sets of oligonucleotide probes comprises N oligonucleotide probes, and wherein each of said oligonucleotide probes comprises a sequence of X bases and at least one detectable label;
(c) contacting said at least one sample with said corresponding sets of oligonucleotide probes so that each of said at least two target nucleic acid sequences is contacted with the corresponding set of oligonucleotide probes;
(d) incubating said at least one sample and said corresponding sets of oligonucleotide probes under conditions that allow hybridization of each of said target nucleic acid sequences, if present in said at least one sample, to said corresponding set of oligonucleotide probes; and
(e) detecting said hybridization, wherein said detecting of said hybridization indicates the relative amounts of each of said at least two target nucleic acid sequences in said at least one sample.

2. The method of claim 1, wherein said at least two target nucleic acid sequences comprise DNA.

3. The method of claim 1, wherein said at least two target nucleic acid sequences comprise RNA.

4. The method of claim 1, wherein said oligonucleotide probes comprise DNA.

5. The method of claim 1, wherein said oligonucleotide probes comprise RNA.

6. The method of claim 1, wherein said oligonucleotide probes comprise a nucleic acid mimic.

7. The method of claim 1, wherein N is identical between each said corresponding set of oligonucleotide probes.

8. The method of claim 1, wherein N comprises between about 2 to about 24 oligonucleotide probes.

9. The method of claim 1, wherein N comprises between about 2 to about 14 oligonucleotide probes.

10. The method of claim 1, wherein N comprises between about 3 to about 10 oligonucleotide probes.

11. The method of claim 1, wherein X comprises between about 15 to about 60 bases.

12. The method of claim 1, wherein X comprises between about 20 to about 50 bases.

13. The method of claim 1, wherein said at least one detectable label comprises at least one detectable label selected from the group consisting of a radioactive isotope, a non-radioactive isotope, a fluorophore, a luminophore, a dye, a pigment, at least one member of a resonance transfer pair, a particle, an enzyme, an antigenically recognizable structure, a bindable moiety, and a combination thereof.

14. The method of claim 1, wherein each said oligonucleotide probe comprises a GC content of between about 40% to about 65%.

15. The method of claim 1, wherein each said oligonucleotide probe comprises a GC content of between about 45% to about 60%.

16. The method of claim 1, wherein each said oligonucleotide probe comprises a sequence that permits a spacing of at least about 15 bases between adjacent oligonucleotide probes when said adjacent oligonucleotide probes are hybridized to the corresponding said target nucleic acid sequence.

17. The method of claim 1, wherein said hybridization occurs substantially simultaneously for all of said at least two target nucleic acid sequences.

18. The method of claim 1, wherein said hybridization occurs sequentially for said at least two target nucleic acid sequences.

19. The method of claim 1, wherein said detecting of said hybridization quantitatively indicates the relative amounts of each of said at least two target nucleic acid sequences.

20. The method of claim 1, wherein said detecting of said hybridization semi-quantitatively indicates the relative amounts of each of said at least two target nucleic acid sequences.

21. The method of claim 1, wherein said detecting of said hybridization qualitatively indicates the relative amounts of each of said at least two target nucleic acid sequences.

22. A method to detect the relative amounts of at least two target messenger RNAs in at least one microscopy sample, comprising
(a) providing at least one microscopy sample suspected of containing said at least two target messenger RNAs;
(b) providing a corresponding set of oligonucleotide probes for each of said at least two target messenger RNAs, wherein each of said corresponding sets of oligonucleotide probes comprises N oligonucleotide probes, where N comprises between about 2 to about 24 oligonucleotide probes, and wherein each of said oligonucleotide probes comprises
(i) a sequence comprising X bases, where X comprises between about 15 to about 60 bases, and comprising a GC content of between about 40% to about 65%, and permitting a spacing of at least about 15 bases between adjacent oligonucleotide probes when said adjacent oligonucleotide probes are hybridized to the corresponding said target messenger RNA; and
(ii) at least one detectable label;
(c) contacting said at least one microscopy sample with said corresponding sets of oligonucleotide probes so that each of said at least two target messenger RNAs is contacted with the corresponding set of oligonucleotide probes;
(d) incubating said at least one microscopy sample and said corresponding sets of oligonucleotide probes under conditions that allow in situ hybridization of each of said target messenger RNAs, if present in said at least one microscopy sample, to said corresponding set of oligonucleotide probes; and
(e) detecting said in situ hybridization, wherein said detecting of said in situ hybridization indicates the relative amounts of each of said at least two target messenger RNAs in said at least one microscopy sample.

23. A method to provide sets of oligonucleotide probes useful in detecting at least two target nucleic acid sequences, comprising the steps of:
(a) selecting said at least two target nucleic acid sequences;
(b) designing a corresponding set of oligonucleotide probes for each of said at least two target nucleic acid sequences, wherein each of said corresponding sets of oligonucleotide probes comprises N oligonucleotide probes, where N comprises between about 2 to about 24 oligonucleotide probes, and wherein each of said oligonucleotide probes comprises:
(i) a sequence specifically complementary to at least part of corresponding said target nucleic acid sequence, and comprising X bases, where X comprises between about 15 to about 60 bases, and comprising a GC content of between about 40% to about 65%, and permitting a spacing of at least about 15 bases between adjacent oligonucleotide probes when said adjacent oligonucleotide probes are hybridized to the corresponding said target nucleic acid sequence; and
(ii) at least one detectable label; and
(c) synthesizing said designed corresponding sets of oligonucleotide probes for each of said at least two target nucleic acid sequences.


SEQUENCE LISTING (20 sequences)

SEQ ID NO: 1 - RNA, 1415 nt, artificial sequence
  synthetic construct
aggaacggccacgcuguagggucucugcuugaaugugccggcugucuggguggggaaugcccgggaggacgugccguagucaggaggggggagggccaggucauccuugucuaggagguugccugugcugcugcucuugcccugcugcaggcucauaugcaaucugucacuuccgucacuguccaggacccggguguaggagaaggggaaccagccccgcaucuuggucuucucacucuccccauagugccagccgucacgggccucaggcacuagcagcgugaugaggucgcccuccuugaagcucagcagagugcuauugucaccggccgcgugggagaaaauggccuugacccgcaugcggccguuacguucuaggccagcugccauggagcuugaacguggcagugucuuguucucagugguggcauagcuguucuucggcgucacgcucuugcgcacggggaguguguuggaguacgagucgcucagcuugcuuugagacugcgggggagacagggauuugggcugggcagccuuucggucagcccagggguuguaguccucgcuguccgggccugcgacgccauucaugacagguacauucuccugagcagacauccgccccacaaauggugccagcucagguggcacuggcaagggcuuggcuccaggaaugggaucugagaugaccagguuggacuuggaagcugacagggcacuuggaaggauggagccauugcuauuggccaucuguugcaucagcuggacagcacggucugggaucuuguuggggucggcacaggcuugcugccacagaggcagcuucugggccagcaacuccuugcccuuggaaugguaagcagcagaguucuuggccacagcgcacugcuuuuccaccaggaagcagaaccuccugcgcuccucagugagugcugucuuguagccgucagacacguaguucuccagcucgcccugcuuauugcugauggcaucgauguacugcagcuccuugucugaguacuucugaggguucuuacucccuuggcucuucuugcggagcuucuucagcucagccugacacuuguccagggcguccccuuugcuccucuguuccguuugguauuucuucagugcagcacuuagauaccuggaguccaguucuacuuucugcuccagcugcgugagcagcucauugugaaaagacuucagcgucucuuccaacugguucuggaucugccggugcaccucagccaucuggaagaggacguccccaaguuccuuagagcccuggcucucgcuggccagcucccccaucuuuaccagagcaucgaaauagccuuuggcagcgaaggugacaccugccagugcuuucucauaguucuugcccauggcgaugaaguugcggaggcugg

SEQ ID NO: 2 - DNA, 40 bp, artificial sequence
  synthetic construct
tccgagcgtgaaagcgacatggtcctgggtcccggctaca

SEQ ID NO: 3 - DNA, 40 bp, artificial sequence
  synthetic construct
cccatctttaccagagcatcgaaatagcctttggcagcga

SEQ ID NO: 4 - DNA, 40 bp, artificial sequence
  synthetic construct
tcgccctgcttattgctgatggcatcgatgtactgcagct

SEQ ID NO: 5 - DNA, 40 bp, artificial sequence
  synthetic construct
gaatgggatctgagatgaccaggttggacttggaagctga

SEQ ID NO: 6 - DNA, 40 bp, artificial sequence
  synthetic construct
tagcagcgtgatgaggtcgccctccttgaagctcagcaga

SEQ ID NO: 7 - DNA, 40 bp, artificial sequence
  synthetic construct
gctcttgccctgctgcaggctcatatgcaatctgtcactt

SEQ ID NO: 8 - DNA, 21 bp, artificial sequence
  synthetic construct
gccagggctctaaggaacttg

SEQ ID NO: 9 - DNA, 20 bp, artificial sequence
  synthetic construct
gcacctcagccatctggaag

SEQ ID NO: 10 - RNA, 743 nt, artificial sequence
  synthetic construct
gacguucucaaacagcugcauggaguucgugauguucucauccuucugacaagucuccagaaauucaucaauggucaccacuccauccugguuccuguccauuuucugaaagaaccucuccacaugcuccaggggugcauccucccgcaggaugggguagguguggcggcccaucaugucguagauggacuucaugauggccagcaucuccuccuuggugaugcaaccauccuuguuaaugucauagagauugaaggcccacuugagcuucucauggaccgucccucgaagcaggauggagagcccaaccacaaaguccucaaaguggauggccccguucccaucagcaucgaaggcauugaagaggaagugugcauaggugguggcaucucccugagggaagaacugggaauaaaugaguuugaaggugucuucauccaccaggccugugggacacucauucuugaagccucgguaaagggacugcagcuccuucuuggugaacuuggucugagcuuguagcugguccaagcccucuggcugauggcgcaccguggauaacucuaguucacugucacugcugucugagccuuguggggcagcacuggacaggauccacuugauuaagcagcaacgcaucagggccuggcgggugaaccguggccuuugccacuugaugcuuucccucuugcucagugguaugcgcccaggaucucccaggagguugccaucugaugccuuca

SEQ ID NO: 11 - DNA, 40 bp, artificial sequence
  synthetic construct
tgccatctgatgccttcacggcttccttggtcctctgcat

SEQ ID NO: 12 - DNA, 40 bp, artificial sequence
  synthetic construct
tggggcagcactggacaggatccacttgattaagcagcaa

SEQ ID NO: 13 - DNA, 40 bp, artificial sequence
  synthetic construct
attcttgaagcctcggtaaagggactgcagctccttcttg

SEQ ID NO: 14 - DNA, 40 bp, artificial sequence
  synthetic construct
cccgttcccatcagcatcgaaggcattgaagaggaagtgt

SEQ ID NO: 15 - DNA, 40 bp, artificial sequence
  synthetic construct
cccacttgagcttctcatggaccgtccctcgaagcaggat

SEQ ID NO: 16 - DNA, 40 bp, artificial sequence
  synthetic construct
gaaagaacctctccacatgctccaggggtgcatcctcccg

SEQ ID NO: 17 - DNA, 21 bp, artificial sequence
  synthetic construct
ctagagttatccacggtgcgc

SEQ ID NO: 18 - DNA, 20 bp, artificial sequence
  synthetic construct
ttgtagctggtccaagccct

SEQ ID NO: 19 - DNA, 20 bp, artificial sequence
  synthetic construct
ccctcacaatttccatccca

SEQ ID NO: 20 - DNA, 20 bp, artificial sequence
  synthetic construct
tccctaggcccctcctgtta